SEQUENCE LISTING (59 sequences)

SEQ ID NO: 1 - DNA, 69 bp, Triticum monococcum
cctttaaaaacccctccccccctgccggaaccctcgttttggcctggccatcctccctctcctcccctc

SEQ ID NO: 2 - DNA, 49 bp, Triticum monococcum
cctttaaaaaccctcgttttggcctggccatcctccctctcctcccctc

SEQ ID NO: 3 - DNA, 35 bp, Triticum monococcum
ccttttggcctggccatcctccctctcctcccctc

SEQ ID NO: 4 - DNA, 21 bp, Triticum monococcum
cctttaaaaacccctcccctc

SEQ ID NO: 5 - DNA, 735 bp, Triticum monococcum
atggggcgcgggaaggtgcagctgaagcggatcgagaacaagatcaaccggcaggtgaccttctccaagcgccgctcggggcttctcaagaaggcgcacgagatctccgtgctctgcgacgccgaggtcggcctcatcatcttctccaccaagggaaagctctacgagttctccaccgagtcatgtatggacaaaattcttgaacggtatgagcgctattcttatgcagaaaaggttctcgtttcaagtgaatctgaaattcagggaaactggtgtcacgaatataggaaactgaaggcgaaggttgagacaatacagaaatgtcaaaaacatctcatgggagaggatcttgaatctttgaatctcaaggagttgcagcaactggagcagcagctggaaagctcactgaaacatatcagatccaggaagaaccaacttatgcacgaatccatttctgagctgcagaagaaggagaggtcactgcaggaggagaataaagttctccagaaggaactcgtggagaagcagaaggcccatgcggcgcagcaagatcaaactcagcctcaaaccagctcttcttcttcttccttcatgctgagggatgctccccctgccgcaaataccagcattcatccagcggcggcaggcgagagggcagaggatgcggcagtgcagccgcaggccccaccccggacggggcttccaccgtggatggtgagccacatcaacgggtga

SEQ ID NO: 6 - DNA, 734 bp, Triticum monococcum
atggggcgcgggaaggtgcagctgaagcggatcgagaacaagatcaaccggcaggtgaccttctccaagcgccgctcggggcttctcaagaaggcgcacgagatctccgtgctctgcgacgccgaggtcggcctcatcatcttctccaccaagggaaagctctacgagttctccaccgagtcatgtatggacaaaattcttgaacggtatgagcgctattcttatgcagaaaaggttctcgtttcaagtgaatctgaaattcagggaaactggtgtcacgaatataggaaactgaaggcgaaggttgagacaatacagaaatgtcaaaaacatctcatgggagaggatcttgaatctttgaatctcaaggagttgcagcaactggagcagcagctggaaagctcactgaaacatatcagatccaggaagaaccaacttatgcaggatccatttctgagctgcagaagaaggagaggtcactgcaggaggagaataaagttctccagaaggaactcgtggagaagcagaaggcccatgcggcgcagcaagatcaaactcagcctcaaaccagctcttcttcttcttccttcatgctgagggatgctccccctgccgcaaataccagcattcatccagcggcggcaggcgagagggcagaggatgcggcagtgcagccgcaggccccaccccggacggggcttccaccgtggatggtgagccacatcaacgggtga

SEQ ID NO: 7 - protein, 244 aa, Triticum monococcum
MGRGKVQLKRIENKINRQVTFSKRRSGLLKKAHEISVLCDAEVGLIIFSTKGKLYEFSTESCMDKILERYERYSYAEKVLVSSESEIQGNWCHEYRKLKAKVETIQKCQKHLMGEDLESLNLKELQQLEQQLESSLKHIRSRKNQLMHESISELQKKERSLQEENKVLQKELVEKQKAHAAQQDQTQPQTSSSSSSFMLRDAPPAANTSIHPAAAGERAEDAAVQPQAPPRTGLPPWMVSHING

SEQ ID NO: 8 - protein, 244 aa, Triticum monococcum
MGRGKVQLKRIENKINRQVTFSKRRSGLLKKAHEISVLCDAEVGLIIFSTKGKLYEFSTESCMDKILERYERYSYAEKVLVSSESEIQGNWCHEYRKLKAKVETIQKCQKHLMGEDLESLNLKELQQLEQQLESSLKHIRSRKNQLMHGSISELQKKERSLQEENKVLQKELVEKQKAHAAQQDQTQPQTSSSSSSFMLRDAPPAANTSIHPAAAGERAEDAAVQPQAPPRTGLPPWMVSHING

SEQ ID NO: 9 - DNA, 1017 bp, Triticum monococcum
atttgcctgatgagacgcttgacaacagtgtattgatggatgtctggtcggtatacacgcacagcacagtacccctactcctaggactggcgagtatctttcattcattccagaaatacgcgggtcggccaaaagtagaaaaatacactgcgcccactcaatccacgcagcgcactgcactgcacagcaacgcttcatgtcaaaagtcgagctcaagcatgcacgcgatggacgcggcgcgaatgacccgggcggcacgacgcgagtgcccgccgcgcccgcccgcctgccccgcagccgacctcttcccaaacgggacaagcgagacggcccaaaacgagcaaggaaagcagcctcctactgtggcagcccgcccccacgaccgtcatctcgccttccattccattttccctggacggaccagacccgtccgagccgccctgacctagccagccagcatttcctctttcgtcccccgccgccgtgaccaaaaaagcaaaaaaggaaaaagggaaaatgctaaaggaaaaaactccgctctttcccttcttctaggcctagggtacagtagaatattataaaaggaaaaattctgctcgttttttgctctgtggtgtgtgtttgtggcgagagaaaatgatttggggaaagcaaaatcgggagattcgcacgtacgatcgttcgacacgtcgacgcccggcgggcccgtggtggggcatcgtgtggctgcaggaccgcggggccccgcggggcgggccgggccaatgggtgctcgacagcggctatgctccagaccagcccggtattgcataccgcgctcggggccagatccctttaaaaaccctcgttttggcctggccatcctccctctcctcccctctcttccacctcacccaaccacctgatagccatggctccgccgcctcgcctccgcctgcgccagtcggagtagccgtcgcggtctgcgggtgttggagggtaggggcgtagggttggcccggttctcgagcggagatg

SEQ ID NO: 10 - DNA, 1036 bp, Triticum monococcum
atttgcctgatgagacgcttgacaacagtgtattgatggatgtctggtcggtatacacgcacagcacagtacccctactcctaggactggcgagtatctttcattcattccagaaatacgcgggtcggccaaaagtagaaaaatacactgcgcccactcaatccacgcagcgcactgcactgcacagcaacgcttcatgtcaaaagtcgagctcaagcatgcacgcgatggacgcggcgcgaatgacccgggcggcacgacgcgagtgcccgccgcgcccgcccgcctgccccgcagccgacctctcccaaacgggacaagcgagacggcccaaaacgagcaaggaaagcagcctcctactgtggcagcccgcccccacgaccgtcatctcgccttccattccattttccctggacggaccagacccgtccgagccgccctgacctagccagccagcatttcctctttcgtcccccgccgccgtgaccaaaaaagcaaaaaaggaaaaagggaaaatgctaaaggaaaaaactccgctctttcccttcttctaggcctagggtacagtagaatattataaaaggaaaaattctgctcgttttttgctctgtggtgtgtgtttgtggcgagagaaaatgatttggggaaagcaaaatcgggagattcgcacgtacgatcgttcgacacgtcgacgcccggcgggcccgtggtggggcatcgtgtggctgcaggaccgcggggccccgcggggcgggccgggccaatgggtgctcgacagcggctatgctccagaccagcccggtattgcataccgcgctcggggccagatccctttaaaaacccctccccccctgccggaaccctcgttttggcctggccatcctccctctcctcccctctcttccacctcacccaaccacctgatagccatggctccgccgcctcgcctccgcctgcgccagtcggagtagccgtcgcggtctgcgggtgttggagggtaggggcgtagggttggcccggttctcgagcggagatg

SEQ ID NO: 11 - DNA, 1036 bp, Triticum monococcum
atttgcctgatgagacgcttgacaacagtgtattgatggatgtctggtcggtatacacgcacagcacagtacccctactcctaggactggcgagtatctttcattcattccagaaatacgcgggtcggccaaaagtagaaaaatacactgcgcccactcaatccacgtagcgcactgcactgcacagcaacgcttcatgtcaaaagtcgagctcaagcatgcacgcgatggacgcggcgcgaatgacccgggcggcacgacgcgagtgcccgccgcgcccgcccgcctgccccgcagccgacctctcccaaacgggacaagcgagacggcccaaaacgagcaaggaaagcagcctcctactgtggcagcccgcccccacgaccgtcatctcaccctccattccattttccctggacggaccagacccgtccgagccgccctgacctagccagccagcatttcctctttcgtcccccgccgccgtgaccaaaaaagcaaaaaaggaaaaagggaaaatgctaaaggaaaaaactccgctctttcccttcttctaggcctagggtacagtagaatattataaaaggaaaaattctgctcgttttttgctctgtggtgtgtgtttgtggcgagagaaaatgatttggggaaagcaaaatcgggagattcgcacgtacgatcgttcgacacgtcgacgcccggcgggcccgtggtggggcatcgtgtggctgcaggaccgcggggccccgcggggcgggccgggccaatgggtgctcgacagcggctatgctccagaccagcccggtattgcataccgcgctcggggccagatccctttaaaaacccctccccccctgccggaaccctcgttttggcctggccatcctccctctcctcccctctcttccacctcacccaaccacctgatagccatggctccgccgcctcgcctccgcctgcgccagtcggagtagccgtcgcggtctgcgggtgttggagggtaggggcgtagggttggcccggttctcgagcggagatg

SEQ ID NO: 12 - DNA, 1036 bp, Triticum monococcum
atttgcctgatgagacgcttgacaacagtgtattgatggatgtctggtcggtatacacgcacagcacagtacccctactcctaggactggcgagtatctttcattcattccagaaatacgcgggtcggccaaaagtagaaaaatacactgcgcccactcaatccacgtagcgcactgcactgcacagcaacgcttcatgtcaaaagtcgagctcaagcatgcacgcgatggacgcggcgcgaatgacccgggcggcacgacgcgagtgcccgccgcgcccgcccgcctgccccgcagccgacctctcccaaacgggacaagcgagacggcccaaaacgagcaaggaaagcagcctcctactgtggcagcccgcccccacgaccgtcatctcaccttccattccattttccctggacggaccagacccgtccgagccgccctgacctagccagccagcatttcctctttcgtcccccgccgccgtgaccaaaaaagcaaaaaaggaaaaagggaaaatgctaaaggaaaaaactccgctctttcccttcttctaggcctagggtacagtagaatattataaaaggaaaaattctgctcgttttttgctctgtggtgtgtgtttgtggcgagagaaaatgatttggggaaagcaaaatcgggagattcgcacgtacgatcgttcgacacgtcgacgcccggcgggcccgtggtggggcatcgtgtggctgcaggaccgcggggccccgcggggcgggccgggccaatgggtgctcgacagcggctatgctccagaccagcccggtattgcataccgcgctcggggccagatccctttaaaaacccctccccccctgccggaaccctcgttttggcctggccatcctccctctcctcccctctcttccacctcacccaaccacctgatagccatggctccgccgcctcgcctccgcctgcgccagtcggagtagccgtcgcggtctgcgggtgttggagggtaggggcgtagggttggcccggttctcgagcggagatg

SEQ ID NO: 13 - DNA, 317 bp, Triticum monococcum
gtgtggctgcaggaccgcggggccccgcggggcgggccgggccaatgggtgctcgacagcggctatgctccagaccagcccggtattgcataccgcgctcggggccagatccctttaaaaacccctccccccctgccggaaccctcgttttggcctggccatcctccctctcctcccctctcttccacctcacccaaccacctgatagccatggctccgccgcctcgcctccgcctgcgccagtcggagtagccgtcgcggtctgcgggtgttggagggtaggggcgtagggttggcccggttctcgagcggagatg

SEQ ID NO: 14 - DNA, 297 bp, Triticum monococcum
gtgtggctgcaggaccgcggggccccgcggggcgggccgggccaatgggtgctcgacagcggctatgctccagaccagcccggtattgcataccgcgctcggggccagatccctttaaaaaccctcgttttggcctggccatcctccctctcctcccctctcttccacctcacccaaccacctgatagccatggctccgccgcctcgcctccgcctgcgccagtcggagtagccgtcgcggtctgcgggtgttggagggtaggggcgtagggttggcccggttctcgagcggagatg

SEQ ID NO: 15 - DNA, 269 bp, Triticum monococcum
gtgtggctgcaggaccgcggggccccgcggggcgggccgggccaatgggtgctcgacagcggctatgctccagaccagcccggtattgcataccgcgctcggggccagatccctttaaaaacccctcccctctcttccacctcacccaaccacctgatagccatggctccgccgcctcgcctccgcctgcgccagtcggagtagccgtcgcggtctgcgggtgttggagggtaggggcgtagggttggcccggttctcgagcggagatg

SEQ ID NO: 16 - DNA, 283 bp, Triticum monococcum
gtgtggctgcaggaccgcggggccccgcggggcgggccgggccaatgggtgctcgacagcggctatgctccagaccagcccggtattgcataccgcgctcggggccagatcccttttggcctggccatcctccctctcctcccctctcttccacctcacccaaccacctgatagccatggctccgccgcctcgcctccgcctgcgccagtcggagtagccgtcgcggtctgcgggtgttggagggtaggggcgtagggttggcccggttctcgagcggagatg

SEQ ID NO: 17 - DNA, 316 bp, Triticum monococcum
gtgtggctgcaggaccgcggggccccgcggggcgggccgggccaatgggtgctcgacagcggctatgctccagaccagcccggtattgcataccgcgctcggggccagatccctttaaaaacccctccccccctgccggaaccctgttttggcctggccatcctccctctcctcccctctcttccacctcacccaaccacctgatagccatggctccgccgcctcgcctccgcctgcgccagtcggagtagccgtcgcggtctgcgggtgttggagggtaggggcgtagggttggcccggttctcgagcggagatg

SEQ ID NO: 18 - DNA, 735 bp, Hordeum vulgare
atggggcgcaggaaggtgcagctgaagcggatcgagaacaagatcaaccgccaggtcaccttctccaagcgccgctcggggctgctcaagaaggcgcacgagatctccgtgctctacgacgccgaggtcggcctcatcatcttctccaccaagggaaagctctacgagttctccaccgagtcatgtatggacaaaattcttgaacggtatgagcgctactcttatgcagaaaaggttctcgtttcaagtgaatctgaaattcagggaaactggtgtcacgaatataggaaactgaaggcgaaggttgagacaatacagaaatgtcaaaagcatctcatgggagaggatcttgaatctttgaatctcaaggagttgcagcaactggagcagcagctggaaagctcactgaaacatatcagagccaggaagaaccaacttatgcacgaatccatttctgagcttcagaagaaggagaggtcactgcaggaggagaataaagttctccagaaggaacttgtggagaagcagaaggcccaggcggcgcagcaagatcaaactcagcctcaaaccagctcttcttcttcttccttcatgatgagggatgctccccctgtcgcagataccagcaatcacccagcggcggcaggcgagagggcagaggatgtggcagtgcagcctcaggtcccactccggacggcgcttccactgtggatggtgagccacatcaacggctga

SEQ ID NO: 19 - protein, 244 aa, Hordeum vulgare
MGRRKVQLKRIENKINRQVTFSKRRSGLLKKAHEISVLYDAEVGLIIFSTKGKLYEFSTESCMDKILERYERYSYAEKVLVSSESEIQGNWCHEYRKLKAKVETIQKCQKHLMGEDLESLNLKELQQLEQQLESSLKHIRARKNQLMHESISELQKKERSLQEENKVLQKELVEKQKAQAAQQDQTQPQTSSSSSSFMMRDAPPVADTSNHPAAAGERAEDVAVQPQVPLRTALPLWMVSHING

SEQ ID NO: 20 - protein, 244 aa, Triticum monococcum
MGRGKVQLKRIENKINRQVTFSKRRSGLLKKAHEISVLCDAEVGLIIFSTKGKLYEFSTESCMDKILERYERYSYAEKVLVSSESEIQGNWCHEYRKLKAKVETIQKCQKHLMGEDLESLNLKELQQLEQQLESSLKHIRSRKNQLMHESISELQKKERSLQEENKVLQKELVEKQKAQAAQQDQTQPQTSSSSSSFMMRDAPPAAATSIHPAAAGERAGDAAVQPQAPPRTGLPLWMVSHING

SEQ ID NO: 21 - protein, 245 aa, Lolium temulentum
MGRGKVQLKRIENKINRQVTFSKRRSGLLKKAHEISVLCDAEVGLIIFSTKGKLYEFATDSCMDKILERYERYSYAEKVLISTESEIQGNWCHEYRKLKAKVETIQRCQKHLMGEDLESLNLKELQQLEQQLESSLKHIRSRKSQLMHESISELQKKERSLQEENKILQKELIEKQKAHTQQAQLEQTQPQTSSSSSSFMMGEATPATNRSNPPAAASDRAEDATGQPPARTVLPPWMVSHLNNG

SEQ ID NO: 22 - DNA, 1207 bp, Lolium temulentum
ctctcttcttccccactggacgcacgccatgacaccggccccacggctccacctgcaccctcgggactagccgtcgccgtcgccgtccgggcgggttgtcgattagggtttggtctgctcttccagggagggaggcgagatggggcgcggcaaggtgcagctcaagcggatcgagaacaagatcaaccgccaggtcaccttctccaagcgccgctcaggcctgctcaagaaggcgcacgagatctccgtgctctgcgacgcagaggtcgggctcatcatcttctccaccaagggaaagctctacgagttcgccaccgactcatgtatggacaaaattcttgagcggtatgagcgctactcctatgcagagaaagtgctcatttcaactgaatctgaaattcagggaaactggtgtcatgaatataggaaactgaaggcgaaggttgagacaatacagagatgtcaaaagcatctaatgggagaggatcttgaatcattgaatctcaaggagttgcagcaactagagcagcagctggaaagttcactgaaacatattagatccagaaagagccagcttatgcacgaatccatatctgagcttcaaaagaaggagaggtcactgcaagaggagaataaaattctccagaaggaactcatagagaagcagaaggcccacacgcagcaagcgcagttggagcaaactcagccccaaaccagctcttcctcctcctcctttatgatgggggaagctaccccagcaacaaatcgcagtaatcccccagcagcggccagcgacagagcagaggatgcgacggggcagcctccagctcgcacggtgcttccaccatggatggtgagtcacctcaacaatggctgaagggtccttccactccatctaaacgtattattcagtacgtgtagcgagctgcaccggcctgtcttgtggttgcctagcaagctgaccctcctgcgtgagctgacttcacgtaaggtagcaggttgcaatgtgtatatttcactctgttctgctcagtttccctcctgcgtgagctgacttcacgtaagagttatttaacttgtaatacatgtgtagcgtgagtgacaaattttccactttctacgaccctcttgggtaccgtctgtttctgtgaattaaactatccaatatcagtattatgtatattgtgattgttgaaaaaaaaaaaaa

SEQ ID NO: 23 - DNA, 11 bp, T. monococcum
cctcgttttgg

SEQ ID NO: 24 - DNA, 20 bp, Artificial Sequence
  Primer
ccagcgtatgatttggaggt

SEQ ID NO: 25 - DNA, 20 bp, Artificial Sequence
  Primer
ttggcattattggaccatca

SEQ ID NO: 26 - DNA, 20 bp, Artificial Sequence
  Primer
ctgacctggggccttgagag

SEQ ID NO: 27 - DNA, 20 bp, Artificial Sequence
  Primer
cttcgcatcagcagctctat

SEQ ID NO: 28 - DNA, 20 bp, Artificial Sequence
  Primer
ccatggataatcatcgggag

SEQ ID NO: 29 - DNA, 20 bp, Artificial Sequence
  Primer
gtcaccatcaccaacttcaa

SEQ ID NO: 30 - DNA, 20 bp, Artificial Sequence
  Primer
gactgcgtatttggacgacc

SEQ ID NO: 31 - DNA, 21 bp, Artificial Sequence
  Primer
ccacggctgatatcccgactg

SEQ ID NO: 32 - DNA, 19 bp, Artificial Sequence
  Primer
gaccctcgagaggtaccag

SEQ ID NO: 33 - DNA, 20 bp, Artificial Sequence
  Primer
catctacactacgatctagc

SEQ ID NO: 34 - DNA, 22 bp, Artificial Sequence
  Primer
gaaaatgtctgaacaagctgct

SEQ ID NO: 35 - DNA, 20 bp, Artificial Sequence
  Primer
tctagatgagcaatctgcat

SEQ ID NO: 36 - DNA, 20 bp, Artificial Sequence
  Primer
gaagatgcacttggagaagg

SEQ ID NO: 37 - DNA, 20 bp, Artificial Sequence
  Primer
gtctctttgcattgtaccca

SEQ ID NO: 38 - DNA, 20 bp, Artificial Sequence
  Primer
ggtaaaagatgagcaaggag

SEQ ID NO: 39 - DNA, 25 bp, Artificial Sequence
  Primer
tctatctatggtgaactcttacttc

SEQ ID NO: 40 - DNA, 20 bp, Artificial Sequence
  Primer
ggaaactggtgtcacgaata

SEQ ID NO: 41 - DNA, 20 bp, Artificial Sequence
  Primer
caaggggtcaggcgtgctag

SEQ ID NO: 42 - DNA, 20 bp, Artificial Sequence
  Primer
gaggatttggctccactgag

SEQ ID NO: 43 - DNA, 20 bp, Artificial Sequence
  Primer
tctagggcctggaagaagtg

SEQ ID NO: 44 - DNA, 19 bp, Artificial Sequence
  Primer
atgtggatatcaggaagga

SEQ ID NO: 45 - DNA, 19 bp, Artificial Sequence
  Primer
ctcatacggtcagcaatac

SEQ ID NO: 46 - DNA, 21 bp, Artificial Sequence
  Primer
atggaagctgctggaatccat

SEQ ID NO: 47 - DNA, 36 bp, Artificial Sequence
  Probe
ccttcctgatatccacatcacacttcatgatagagt

SEQ ID NO: 48 - DNA, 24 bp, Artificial Sequence
  Primer
ccttgctcatacggtcagcaatac

SEQ ID NO: 49 - DNA, 354 bp, Triticum monococcum
gagaagagctatgagctgcctgatgggcaggtgatcaccattggggcagagaggttccgttgccctgaggtccttttccagccatctttcattggtatggaagctgctggaatccatgagaccacctacaactctatcatgaagtgtgatgtggatatcaggaaggatctgtatggtaacatcgtgctcagtggtggctcaactatgttcccgggtattgctgaccgtatgagcaaggagatcactgcccttgcaccaagcagcatgaagatcaaggtggtggcaccgcctgagaggaagtacagtgtctggattggagggtcgattcttgcctcccttagtaccttccaacag

SEQ ID NO: 50 - DNA, 23 bp, Artificial Sequence
  Primer
aactcagcctcaaaccagctctt

SEQ ID NO: 51 - DNA, 23 bp, Artificial Sequence
  Probe
catgctgagggatgctccccctg

SEQ ID NO: 52 - DNA, 22 bp, Artificial Sequence
  Primer
ctggatgaatgctggtatttgc

SEQ ID NO: 53 - DNA, 222 bp, Triticum monococcum
ctcgtggagaagcagaaggcccatgcggcgcagcaagatcaaactcagcctcaaaccagctcttcttcttcttccttcatgctgagggatgctccccctgccgcaaataccagcattcatccagcggcggcaggcgagagggcagaggatgcggcagtgcagccgcaggccccaccccggacggggcttccaccgtggatggtgagccacatcaacgggtga

SEQ ID NO: 54 - DNA, 26 bp, Artificial Sequence
  Primer
atgcagatctttgtgaagacccttac

SEQ ID NO: 55 - DNA, 30 bp, Artificial Sequence
  Probe
caagaccatcactctggaggttgagagctc

SEQ ID NO: 56 - DNA, 20 bp, Artificial Sequence
  Primer
gtcctggatcttggccttga

SEQ ID NO: 57 - DNA, 228 bp, Triticum monococcum
atgcagatctttgtgaagacccttactggcaagaccatcactctggaggttgagagctcagacaccatcgacaatgtcaaggccaagatccaggacaaggagggcatccccccggaccagcagcgcctcatcttcgcaggaaagcagctggaggatggccgcactcttgctgactacaacatccagaaggagtccactcttcaccttgtcctgcgtcttcgtggcggt

SEQ ID NO: 58 - DNA, 18 bp, Artificial Sequence
  Primer
acagcggctatgctccag

SEQ ID NO: 59 - DNA, 20 bp, Artificial Sequence
  Primer
tatcaggtggttgggtgagg

(19) United States
(12) Patent Application Publication
(10) Pub. No.: US 2004/0203141 A1
(43) Pub. Date: Oct. 14, 2004

(54) Use of the AP1 gene promoter to control the vernalization response and flowering time in grasses

(76) Inventors: Jorge Dubcovsky, Davis, CA (US); Liuling Yan, Davis, CA (US)

Correspondence Address: Michael R. Ward, Morrison & Foerster LLP, 425 Market Street, San Francisco, CA, 94105-2482, US

(21) Appl. No.: 10/412,137

(22) Filed: Apr. 11, 2003

(51) Int. Cl. C12N 15/82; C12N 15/87; C07H 21/04; C12N 15/63; C12N 5/10; C12N 5/04; A01H 5/00; A01H 5/10

(52) U.S. Cl. 435/320.1; 536/24.1; 435/419; 435/468; 800/278; 536/23.6

(57) ABSTRACT

Winter wheats require several weeks at low temperature to flower. This process called vernalization is controlled mainly by the VRN1 gene. Using 6,190 gametes VRN1 was found to be completely linked to MADS-box genes AP1 and AGLG1 in a 0.03-cM interval flanked by genes Cysteine and Cytochrome B5. No additional genes were found between the last two genes in 324-kb of wheat sequence or in the colinear regions in rice and sorghum. Wheat AP1 and AGLG1 genes were similar to Arabidopsis meristem identity genes AP1 and AGL2 respectively. AP1 transcription was regulated by vernalization in both apices and leaves, and the progressive increase of AP1 transcription was consistent with the progressive effect of vernalization on flowering time. Vernalization was required for AP1 transcription in apices and leaves in winter wheat but not in spring wheat. AGLG1 transcripts were detected during spike differentiation but not in vernalized apices or leaves, suggesting that AP1 acts upstream of AGLG1. No differences were detected between genotypes with different VRN1 alleles in the AP1 and AGLG1 coding regions, but three independent deletions were found in the promoter region of AP1. These results suggest that AP1 is a better candidate for VRN1 than AGLG1. The epistatic interactions between vernalization genes VRN1 and VRN2 suggested a model in which VRN2 would repress directly or indirectly the expression of AP1. A mutation in the promoter region of AP1 would result in the lack of recognition of the repressor and in a dominant spring growth habit.

### GOVERNMENT INTEREST

This invention was made with Government support under Grant (or Contract) No. 2000-1678 awarded by the USDA-NRI. The Government has certain rights in the invention.

### FIELD OF THE INVENTION

This invention is in the field of plant breeding and plant molecular biology. In particular, this invention relates to non-naturally occurring plants with an altered response to vernalization.

### BACKGROUND OF THE INVENTION

Little is known about the molecular regulation of the vernalization response in grasses. If the molecular mechanism of the vernalization response was better understood, the response could be engineered to alter a plant's response to vernalization to improve flowering, growth efficiency and, ultimately, yield. There is thus a tremendous need to identify molecular factors involved with a plant's response to vernalization. In particular, there is a need to identify promoters involved in the vernalization response.

### SUMMARY OF THE INVENTION

In order to meet these needs, the present invention is directed to the finding that the AP1 promoter controls the vernalization response in wheat. The “AP1 promoter sequence” as defined herein refers to any sequence that hybridizes to the nucleic acid molecule of SEQ ID NO:12 (FIG. 9) or the complement thereof under high stringency conditions, or any sequence that includes the critical regulatory recognition sites for vernalization present in SEQ ID NO:12, including the CCTCGTTTTGG (SEQ ID NO:23) sequence located −162 to −172 bp upstream from the start codon of the AP1 gene. This 11-bp region will be referred hereafter as the “CarG-box”. The present invention is thus directed to a recombinant AP1 promoter sequence such as those depicted in FIGS. 9A-(b and 11. In particular, the present invention is directed to recombinant AP1 promoters and their use in plant molecular biology and plant breeding. In a first format, the recombinant AP1 promoter sequence with all or a portion of the CarG box may be operably linked to any heterologous protein coding sequence and introduced into a plant to regulate the expression of the protein by vernalization.

In a second format, the AP1 promoter with or without part or all the CarG box may be operably linked to an AP1 protein encoding sequence and introduced into a plant to modify flowering time or the vernalization requirement in the plant. The AP1 protein encoding sequences of the invention include those sequences that hybridize under high stringency conditions to a nucleic acid selected from SEQ ID NO:5, SEQ ID NO:6, SEQ ID NO:18 and SEQ ID NO:22. The AP1 protein coding sequences may encode AP1 proteins such as those having polypeptide sequences selected from SEQ ID NO: 7, 8, 19, 20 and 21.

The recombinant AP1 promoter sequences of the invention may be cloned into a vector. The vector may be introduced into a cell. The cell may be a prokaryotic cell or a eukaryotic cell. In a preferred format, the cell is a plant cell.

The recombinant AP1 promoter sequences may be introduced into a transgenic plant. The transgenic plant may be transgenic wheat, barley, rye, oats, or forage grasses. The invention is further directed to seed from the transgenic plants of the invention.

The present invention is further directed to a method for altering a plant's response to vernalization. A plant's response to vernalization is said to be altered when the requirement of vernalization or the effect of vernalization in the acceleration of flowering is modified by the expression of a heterologous protein in the plant. The method of the invention includes, as a first step, transforming a plant or plant tissue with a genetic construct having a recombinant AP1 promoter sequence operably linked to a recombinant heterologous protein sequence. The AP1 promoter sequence may lack all or a portion of nucleotides −162 to −172 upstream of the start codon of SEQ ID NO:12. In the method of the invention, the recombinant protein sequence may be an AP1 protein-encoding sequence or any other useful heterologous protein. The method includes, as a second step, expressing the genetic construct in the plant to alter the plant's response to vernalization or its flowering time independently of vernalization. The plant may be selected from wheat, barley, rye, oats and forage grasses.

The present invention is further directed to molecular marker for Vrn1 derived from a gene selected from the group of genes depicted in FIG. 1.

### BRIEF DESCRIPTION OF THE DRAWINGS

FIG. 1 depicts physical maps of the VRN1 regions of various plants. A depicts a genetic map of the VRN1 region on chromosome 5Am of T. monococcum. Genetic distances are in cM (6,190 gametes). B-D depict physical maps of the colinear VRN1 regions in rice, sorghum, and wheat. Regions indicated in red have been sequenced. Double dot lines indicate gaps in the current physical maps. B shows the sequence of the colinear region in rice chromosome 3. C shows S. bicolor BACs 170F8 (AF503433) and 17E12 (AY188330). D shows a T. monococcum physical map. BAC clones order from left to right is: 49I16, 115G1, 136F13, 133P9, 116F2, 89E14, 160C18, 491M20, 328O3, 609E6, 393O11, 719C13, 454P4, 54K21, 579P2, 601A24, 231A16, 638J12, 52F19, 242A12, 668L22, 539M19, and 309P20 (bold letters indicate sequenced BACs). Black dots indicate validation of BAC connections by hybridization. E shows the gene structure of two MADS-box genes completely linked to the VRN1 gene (AY188331, AY188333). Bars represent exons. F shows the sequence comparison of the AP1 promoter regions from genotypes carrying the Vrn1 and vrn1 alleles, and from two T. monococcum accessions with additional deletions (SEQ ID NO:1-4). The last two accessions are spring but their genotype has not been determined yet. Numbers indicate distances from the start codon. A putative MADS-box protein-binding site (CArG-box) is highlighted.

FIG. 2A identifies plants with critical crossovers flanking VRN1. A: homozygous for G1777 (vrn1); B: homozygous for G2528 (Vrn1); H: heterozygous. X: crossover between two markers. F₂ Vrn1 genotype inferred from F₃ progeny test. The names of marker genes are the same as indicated in FIG. 1. FIG. 2B depicts Progeny tests for plants with critical recombination events as determined by the closest heterozygous molecular marker to AP1. “N” indicates the number of plants in each class. “D” indicates the range of heading dates of the unvernalized plants with a particular genotype after the heading date of the of control spring parent G2528 used as zero.

FIG. 3 depicts the relationship between wheat proteins AP1 and AGLG1 and other plant MADS-box proteins in a Neighbor-joining tree. Confidence values on the branches are based on 1000 bootstraps. Tm=Triticum monococcum, Hv=Hordeum vulgare, Os=Oryza sativa, At=Arabidopsis thaliana.

FIG. 4A shows a Reverse Transcription Polymerase Chain Reaction (RT PCR) experiment using T. monococcum G3116 (winter growth habit) and AP1, AGLG1 and AC77N specific primers. The PCR reactions for the three genes were performed using the same cDNA samples. Leaves 1-5): Leaves 1) Before vernalization; 2-4) 2, 4 and 6 weeks of vernalization; 5) two weeks after vernalized plants were returned to the greenhouse; 6) unvernalized apices; 7) 6-weeks vernalized apices; 8) young spikes. FIG. 4B shows the AP1 transcription levels in leaves relative to ACTIN measured by quantitative PCR. 1-5) Leaves from plants at the same vernalization stage as samples 1-5 in 4A. Units are linearized values using the 2^{(-ΔΔCT)} method, where CT is the threshold cycle.

FIG. 5 shows AP1 transcription in leaves of different age. The numbers on the x-axis represent different leaves from the same tiller from the youngest (1) to the oldest (5). The numbers on the y-axis represent linearized values using the 2^{(-ΔΔCT)} method, where CT is the threshold cycle

FIG. 6 shows a restriction map of sorghum BAC 17E12. The horizontal lines indicate the fragment detected by hybridization of the Southern blots of the restriction maps with the probe indicated above.

FIG. 7 depicts allelic variation in the wheat AP1 DNA sequences. The bolded and underlined nucleotide indicates the only polymorphism in the coding region. FIG. 7A depicts the sequence of G2528 (vrn1)=DV92 (vrn1) [SEQ ID NO:5]. FIG. 7B depicts the sequence of G1777 (vrn1)=G3116 (vrn1) [SEQ ID NO:6].

FIG. 8 depicts allelic variation in the wheat AP1 protein sequence. The bolded and underlined amino acid indicates a difference in the sequence. FIG. 8A depicts the sequence of G2528 (vrn1) DV92 (vrn1) [SEQ ID NO:7]. FIG. 8B depicts the sequence of G1777(vrn1)=G3116 (vrn1) [SEQ ID NO:8].

FIGS. 9A and 9B depict allelic variation in the AP1 promoter region. G2528: Vrn1 allele [SEQ ID NO:9], DV92=G1777=G3116=vrn1 allele [SEQ ID NO:10-12]; ATTTGCCT End of the 401-bp repetitive element; GA host duplication created by the insertion of he repetitive element Highlighted: differences between Vrn1 and vrn1 genotypes. Underlined: 5′ UTR based on alignment with ESTs BF429319 and BF484655

FIG. 10 depicts a gel showing the 34 and 48 bp deletions in the AP1 promoter region. Lines 1, 3, 4, and 5 (PI355516, PI352473, PI272561, PI573529): cultivated T. monococcum accessions with winter growth habit. Line 2 (PI349049): 34-dp deletion, spring growth habit. Line 6 (PI355515): 48-bp deletion, spring growth habit. Line 7: G3116, vrn1. Lines 8: G1777, vrn1. Line 9: G2528, Vrn1 (20-bp deletion).

FIG. 11 shows the sequence showing the 34 and 48 bp deletions in the AP1 promoter region. Putative regulatory sequences are indicated in bold and underlined (CCAAT-box), (TATA-box), and (CarG-box) letters. G1777 is SEQ ID NO:13; G2528 is SEQ ID NO:14; PI355515 is SEQ ID NO:15; PI349049 is SEQ ID NO:16, PI503874 is SEQ ID NO:17.

FIG. 12 shows a model of the regulation of flowering initiation by vernalization in wheat.

### DETAILED DESCRIPTION OF THE INVENTION

Before explaining at least one embodiment of the invention in detail, it is to be understood that the invention is not limited in its application to the details of construction and the arrangement of the components set forth in the following description. The invention is capable of other embodiments or of being practiced or carried out in various ways. Also, it is to be understood that the phraseology and terminology employed herein is for the purpose of description and should not be regarded as limiting.

Throughout this disclosure, various publications, patents and published patent specifications are referenced. The disclosures of these publications, patents and published patent specifications are hereby incorporated by reference into the present disclosure to more fully describe the state of the art to which this invention pertains. The practice of the present invention will employ, unless otherwise indicated, conventional techniques of plant breeding, immunology, molecular biology, microbiology, cell biology and recombinant DNA, which are within the skill of the art. See, e.g., Sambrook, Fritsch and Maniatis, MOLECULAR CLONING: A LABORATORY MANUAL, 2nd edition (1989); CURRENT PROTOCOLS IN MOLECULAR BIOLOGY [(F. M. Ausubel, et al. eds., (1987)]; PLANT BREEDING: PRINCIPLES AND PROSPECTS (Plant Breeding, Vol 1) M. D. Hayward, N. O. Bosemark, I. Romagosa; Chapman & Hall, (1993.); Coligan, Dunn, Ploegh, Speicher and Wingfeld, eds. (1995) CURRENT PROTOCOLS IN PROTEIN SCIENCE (John Wiley & Sons, Inc.); the series METHODS IN ENZYMOLOGY (Academic Press, Inc.): PCR 2: A PRACTICAL APPROACH (M. J. MacPherson, B. D. Hames and G. R. Taylor eds. (1995)], Harlow and Lane, eds. (1988) ANTIBODIES, A LABORATORY MANUAL, and ANIMAL CELL CULTURE [R. I. Freshney, ed. (1987)].

Unless otherwise noted, technical terms are used according to conventional usage. Definitions of common terms in molecular biology may be found in Lewin, Genes V, published by Oxford University Press, 1994 (SBN 0-19-854287-9); Kendrew et al. (eds.), The Encyclopedia of Molecular Biology, published by Blackwell Science Ltd., 1994 (SBN 0-632-02182-9); and Robert A. Meyers (ed.), Molecular Biology and Biotechnology, a Comprehensive Desk Reference, published by VCH Publishers, Inc., 1995 (ISBN 1-56081-569-8); Ausubel et al. (1987) Current Protocols in Molecular Biology, Green Publishing; Sambrook et al. (1989) Molecular Cloning: A Laboratory Manual, Cold Spring Harbor, N.Y.

In order to facilitate review of the various embodiments of the invention, the following definitions are provided:

AP1 protein or AP1 polypeptide: An AP1 protein or AP1 polypeptide is a protein encoded by the floral meristem identity gene APETALA1 (AP1). In Arabidopisis, mutations in AP1 result in replacement of a few basal flowers by inflorescence shoots that are not subtended by leaves. An apical flower produced in an ap1 mutant has an indeterminate structure in which a flower arises within a flower.

The present invention may be practiced using nucleic acid sequences that encode full length AP1 proteins as well as AP1 derived proteins that retain AP1 activity. AP1 derived proteins which retain AP1 biological activity include fragments of AP1, generated either by chemical (e.g. enzymatic) digestion or genetic engineering means; chemically functionalized protein molecules obtained starting with the exemplified protein or nucleic acid sequences, and protein sequence variants, for example allelic variants and mutational variants, such as those produced by in vitro mutagenesis techniques, such as gene shuffling (Stemmer et al., 1994a, 1994b). Thus, the term “AP1 protein” encompasses full-length AP1 proteins, as well as such AP1 derived proteins that retain AP1 activity.

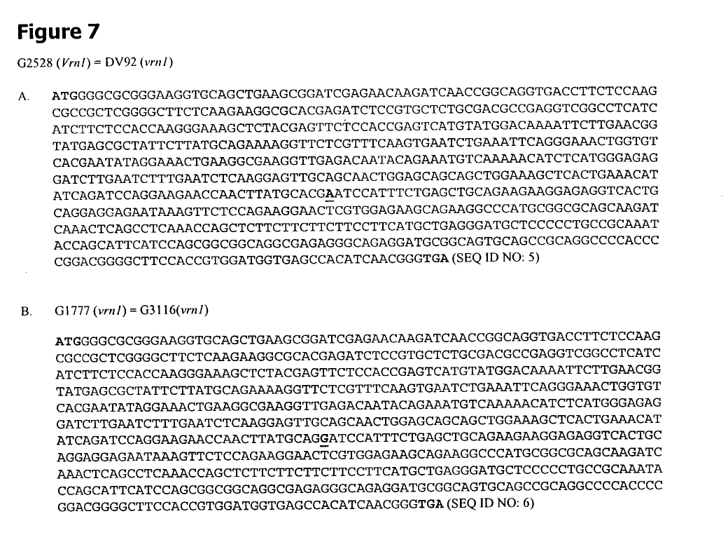

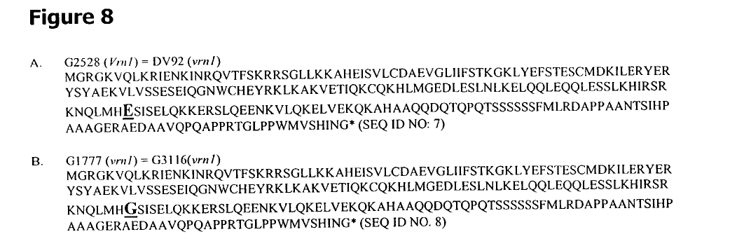

Representative but non-limiting AP1 sequences useful in the invention include the wheat AP1 DNA sequences depicted in FIGS. 7A and 7B and the corresponding protein sequences depicted in FIGS. 8A and 8B.

Also encompassed within the definition of AP1 sequences include the barley AP1 protein (BM5 AJ249144) encoded by the following sequence:

| | |
|---|---|
| ATGGGGCGCAGGAAGGTGCAGCTGAAGCGGATCGAGAACAAGATCAACCGCCAGGTCACCTTCTCCAA | (SEQ ID NO: 18) |
| GCGCCGCTCGGGGCTGCTCAAGAAGGCGCACGAGATCTCCGTGCTCTACGACGCCGAGGTCGGCCTCA | |
| TCATCTTCTCCACCAAGGGAAAGCTCTACGAGTTCTCCACCGAGTCATGTATGGACAAAATTCTTGAA | |
| CGGTATGAGCGCTACTCTTATGCAGAAAAGGTTCTCGTTTCAAGTGAATCTGAAATTCAGGGAAACTG | |
| GTGTCACGAATATAGGAAACTGAAGGCGAAGGTTGAGACAATACAGAAATGTCAAAAGCATCTCATGG | |
| GAGAGGATCTTGAATCTTTGAATCTCAAGGAGTTGCAGCAACTGGAGCAGCAGCTGGAAAGCTCACTG | |
| AAACATATCAGAGCCAGGAAGAACCAACTTATGCACGAATCCATTTCTGAGCTTCAGAAGAAGGAGAG | |
| GTCACTGCAGGAGGAGAATAAAGTTCTCCAGAAGGAACTTGTGGAGAAGCAGAAGGCCCAGGCGGCGC | |
| AGCAAGATCAAACTCAGCCTCAAACCAGCTCTTCTTCTTCTTCCTTCATGATGAGGGATGCTCCCCCT | |
| GTCGCAGATACCAGCAATCACCCAGCGGCGGCAGGCGAGAGGGCAGAGGATGTGGCAGTGCAGCCTCA | |
| GGTCCCACTCCGGACGGCGCTTCCACTGTGGATGGTGAGCCACATCAACGGCTGA | |

The corresponding barley AP1 protein (Hv BM5 CAB97352.1 AJ249144) sequence is:

MGRRKVQLKRIENKINRQVTFSKRRSGLLKKAHEISVLYDAEVGLIIFSTKGKLYEFSTESCMDKILERYERY SYAEKVLVSSESEIQGNWCHEYRKLKAKVETIQKCQKHLMGEDLESLNLKELQQLEQQLESSLKHIRARKN QLMHESISELQKKERSLQEENKVLQKELVEKQKAQMQQDQTQPQTSSSSSSFMMRDAPPVADTSNHPA AAGERAEDVAVQPQVPLRTALPLWMVSHING (SEQ ID NO: 19)

Also encompassed within the definition of AP1 sequences include the hexaploid wheat AP1 protein (Ta AP1 BAA33457 MADS) sequence is:

| | |
|---|---|
| MGRGKVQLKRIENKINRQVTFSKRRSGLLKKAHEISVLCDAEVGLIIFSTKGKLYEFSTESCMDKILERYERY | (SEQ ID NO: 20) |
| SYAEKVLVSSESEIQGNWCHEYRKLKAKVETIQKCQKHLMGEDLESLNLKELQQLEQQLESSLKHIRSRKNQL | |
| MHESISELQKKERSLQEENKVLQKELVEKQKAQAAQQDQTQPQTSSSSSSFMMRDAPPAAATSIHPAAAGERA | |
| GDAAVQPQAPPRTGLPLWMVSHING | |

Included within the definition of AP1 sequences for this invention is the Lolium temulentum AP1 protein sequence which is:

| | |
|---|---|
| MGRGKVQLKRIENKINRQVTFSKRRSGLLKKAHEISVLCDAEVGLIIFSTKGKLYEFATDSCMDKILERYERY | (SEQ ID NO: 21) |
| SYAEKVLISTESEIQGNWCHEYRKLKAKVETIQRCQKHLMGEDLESLNLKELQQLEQQLESSLKHIRSRKSQL | |
| MHESISELQKKERSLQEENKILQKELIEKQKAHTQQAQLEQTQPQTSSSSSSFMMGEATPATNRSNPPAAASD | |
| RAEDATGQPPARTVLPPWMVSHLNNG | |

The corresponding Lolium temulentum AP1 DNA sequence (AF035378) encoding the protein sequence is:

| | |
|---|---|
| CTCTCTTCTTCCCCACTGGACGCACGCCATGACACCGGCCCCACGGCTCCACCTGCACCCTCGGGACTA | (SEQ ID NO: 22) |
| GCCGTCGCCGTCGCCGTCCGGGCGGGTTGTCGATTAGGGTTTGGTCTGCTCTTCCAGGGAGGGAGGCGA | |
| GATGGGGCGCGGCAAGGTGCAGCTCAAGCGGATCGAGAACAAGATCAACCGCCAGGTCACCTTCTCCAA | |
| GCGCCGCTCAGGCCTGCTCAAGAAGGCGCACGAGATCTCCGTGCTCTGCGACGCAGAGGTCGGGCTCAT | |
| CATCTTCTCCACCAAGGGAAAGCTCTACGAGTTCGCCACCGACTCATGTATGGACAAAATTCTTGAGCG | |
| GTATGAGCGCTACTCCTATGCAGAGAAAGTGCTCATTTCAACTGAATCTGAAATTCAGGGAAACTGGTG | |
| TCATGAATATAGGAAACTGAAGGCGAAGGTTGAGACAATACAGAGATGTCAAAAGCATCTAATGGGAGA | |
| GGATCTTGAATCATTGAATCTCAAGGAGTTGCAGCAACTAGAGCAGCAGCTGGAAAGTTCACTGAAACA | |
| TATTAGATCCAGAAAGAGCCAGCTTATGCACGAATCCATATCTGAGCTTCAAAAGAAGGAGAGGTCACT | |
| GCAAGAGGAGAATAAAATTCTCCAGAAGGAACTCATAGAGAAGCAGAAGGCCCACACGCAGCAAGCGCA | |
| GTTGGAGCAAACTCAGCCCCAAACCAGCTCTTCCTCCTCCTCCTTTATGATGGGGGAAGCTACCCCAGC | |
| AACAAATCGCAGTAATCCCCCAGCAGCGGCCAGCGACAGAGCAGAGGATGCGACGGGGCAGCCTCCAGC | |
| TCGCACGGTGCTTCCACCATGGATGGTGAGTCACCTCAACAATGGCTGAAGGGTCCTTCCACTCCATCT | |
| AAACGTATTATTCAGTACGTGTAGCGAGCTGCACCGGCCTGTCTTGTGGTTGCCTAGCAAGCTGACCCT | |
| CCTGCGTGAGCTGACTTCACGTAAGGTAGCAGGTTGCAATGTGTATATTTCACTCTGTTCTGCTCAGTT | |
| TCCCTCCTGCGTGAGCTGACTTCACGTAAGAGTTATTTAACTTGTAATACATGTGTAGCGTGAGTGACA | |
| AATTTTCCACTTTCTACGACCCTCTTGGGTACCGTCTGTTTCTGTGAATTAAACTATCCAATATCAGTA | |
| TTATGTATATTGTGATTGTTGAAAAAAAAAAAAA | |

The coding region start and stop sites are bold and underlined.

The maize and Arabidopsis AP1 sequences are also included within the definition of AP1 protein and are disclosed in U.S. Pat. No. 6,355,863 which is hereby incorporated by reference.

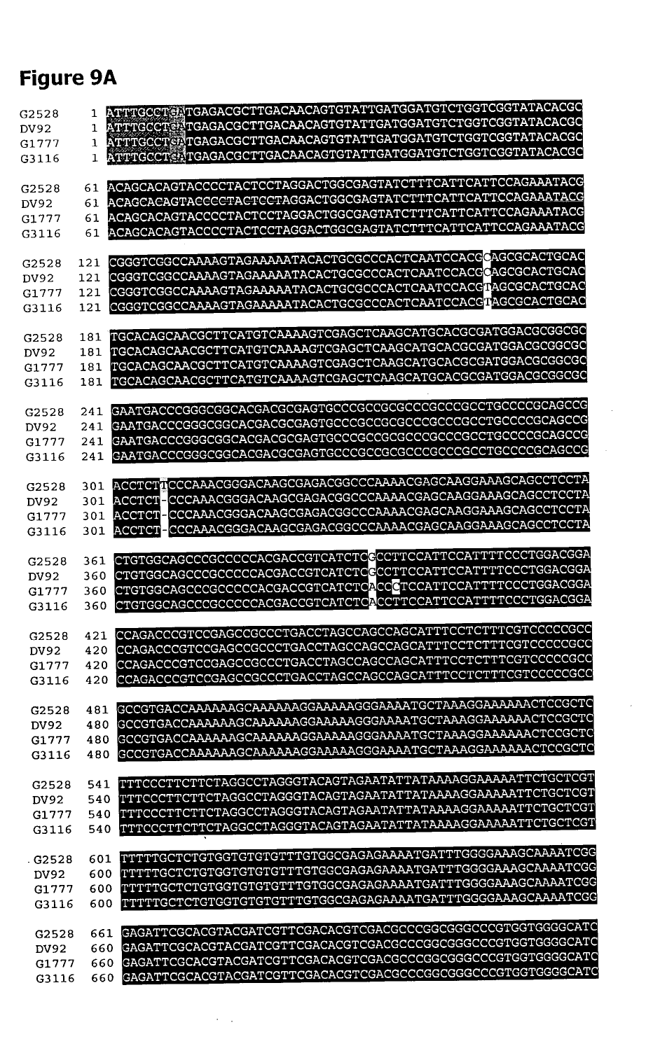

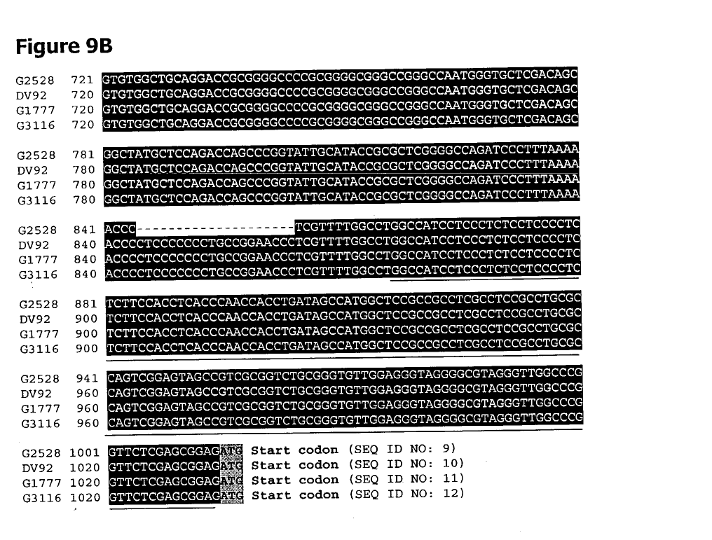

AP1 Promoter: An AP1 promoter is a promoter for the APETALA1 (AP1) gene. AP1 promoters are generally found 5′ to the AP1 protein coding sequence and regulate expression of the AP1 gene. AP1 promoter sequences as defined herein include those sequences that hybridize under high stringency conditions to the nucleic acid of SEQ ID NO:12 (FIG. 9). Such sequences can be synthesized chemically or they can be isolated from plants. AP1 promoters can be spring or winter AP1 promoters, for example, spring wheat or winter wheat AP1 promoters. Representative plants from which AP1 promoters can be isolated include wheat (spring and winter), barley, rye, triticale, oat and forage grasses. A spring AP1 promoter sequence as defined herein includes nucleic acids that hybridize to the nucleic acid molecule of SEQ ID NO:12 or the complement thereof under high stringency conditions wherein the AP1 promoter sequence lacks all or a portion of nucleotides −162 to −172 upstream of the starting ATG, CCTCGTTTTGG (SEQ ID NO:23) or has similar deletions to those indicated in SEQ ID NO: 14, 15, 16 and 17. An AP1 promoter sequence is said to lack all or a portion of SEQ ID NO:23 if 1, 2, 3, 4, 5, 6, 7, 8, 9, 10, 11 of the nucleotides of SEQ ID NO:23 are missing, changed or altered. A winter AP1 promoter sequence as defined herein includes nucleic acids that hybridize to the nucleic acid of SEQ ID NO:12 or the complement thereof under high stringency conditions and that is transcriptionally upregulated by vernalization. Also included in the definition of AP1 promoter are additional natural or synthetic sequences that might not hybridize with SEQ ID NO: 12 but that include the CarG box CCTCGTTTTGG or a related motif that act as a recognition site for the vernalization signal.

Vernalization: Vernalization is the exposure of plants to cold to trigger flowering. For example, winter wheats typically require 4 to 8 weeks at 4° C. to flower.

Sequence Identity: The similarity between two nucleic acid sequences, or two amino acid sequences is expressed in terms of sequence identity (or, for proteins, also in terms of sequence similarity). Sequence identity is frequently measured in terms of percentage identity; the higher the percentage, the more similar the two sequences are. As described herein, homologs and variants of the AP1 nucleic acid molecules may be used in the present invention. Homologs and variants of these nucleic acid molecules will possess a relatively high degree of sequence identity when aligned using standard methods. Such homologs and variants will hybridize under high stringency conditions to one another.

Methods of alignment of sequences for comparison are well known in the art. Various programs and alignment algorithms are described in: Smith and Waterman (1981); Needleman and Wunsch (1970); Pearson and Lipman (1988); Higgins and Sharp (1988); Higgins and Sharp (1989); Corpet et al. (1988); Huang et al. (1992); and Pearson et al. (1994). Altschul et al. (1994) presents a detailed consideration of sequence alignment methods and homology calculations.

The NCBI Basic Local Alignment Search Tool (BLAST) (Altschul et al., 1990) is available from several sources, including the National Center for Biotechnology Information (NCBI, Bethesda, Md.) and on the Internet, for use in connection with the sequence analysis programs blastp, blastn, blastx, tblastn and tblastx. It can be accessed at the NCBI Website. A description of how to determine sequence identity using this program is available at the NCBI website.

Homologs of the disclosed protein and nucleic acid sequences are typically characterized by possession of at least 40% sequence identity counted over the full length alignment with the amino acid sequence of the disclosed sequence using the NCBI Blast 2.0, gapped blastp set to default parameters. The adjustable parameters are preferably set with the following values: overlap span 1, overlap fraction=0.125, word threshold (T)=11. The HSP S and HSP S2 parameters are dynamic values and are established by the program itself depending upon the composition of the particular sequence and composition of the particular database against which the sequence of interest is being searched; however, the values may be adjusted to increase sensitivity. Proteins with even greater similarity to the reference sequences will show increasing percentage identities when assessed by this method, such as at least about 50%, at least about 60%, at least about 70%, at least about 75%, at least about 80%, at least about 90% or at least about 95% sequence identity.

The alignment may include the introduction of gaps in the sequences to be aligned. In addition, for sequences which contain either more or fewer amino acids than the protein encoded by the sequences in the figures, it is understood that in one embodiment, the percentage of sequence identity will be determined based on the number of identical amino acids in relation to the total number of amino acids. Thus, for example, sequence identity of sequences shorter than that shown in the figures as discussed below, will be determined using the number of amino acids in the longer sequence, in one embodiment. In percent identity calculations relative weight is not assigned to various manifestations of sequence variation, such as, insertions, deletions, substitutions, etc.

In one embodiment, only identities are scored positively (+1) and all forms of sequence variation including gaps are assigned a value of “0”, which obviates the need for a weighted scale or parameters as described herein for sequence similarity calculations. Percent sequence identity can be calculated, for example, by dividing the number of matching identical residues by the total number of residues of the “shorter” sequence in the aligned region and multiplying by 100. The “longer” sequence is the one having the most actual residues in the aligned region.

As will be appreciated by those skilled in the art, the sequences of the present invention may contain sequencing errors. That is, there may be incorrect nucleotides, frameshifts, unknown nucleotides, or other types of sequencing errors in any of the sequences; however, the correct sequences will fall within the homology and stringency definitions herein.

High Stringency: High stringency conditions refers to hybridization to filter-bound DNA in 5×SSC, 2% sodium dodecyl sulfate (SDS), 100 ug/ml single stranded DNA at 55-65° C., and washing in 0.1×SSC and 0.1% SDS at 60-65° C.

Vector: A nucleic acid molecule as introduced into a host cell, thereby producing a transformed host cell. A vector may include one or more nucleic acid sequences that permit it to replicate in one or more host cells, such as origin(s) of replication. A vector may also include one or more selectable marker genes and other genetic elements known in the art.

Transformed: A transformed cell is a cell into which has been introduced a nucleic acid molecule by molecular biology techniques. As used herein, the term transformation encompasses all techniques by which a nucleic acid molecule might be introduced into such a cell, plant or animal cell, including transfection with viral vectors, transformation by Agrobacterium, with plasmid vectors, and introduction of naked DNA by electroporation, lipofection, and particle gun acceleration and includes transient as well as stable transformants.

Isolated: An “isolated” biological component (such as a nucleic acid or protein or organelle) has been substantially separated or purified away from other biological components in the cell or the organism in which the component naturally occurs, i.e., other chromosomal and extra-chromosomal DNA and RNA, proteins and organelles. Nucleic acids and proteins that have been “isolated” include nucleic acids and proteins purified by standard purification methods. The term embraces nucleic acids including chemically synthesized nucleic acids and also embraces proteins prepared by recombinant expression in vitro or in a host cell and recombinant nucleic acids as defined below.

Operably linked: A first nucleic acid sequence is operably linked with a second nucleic acid sequence when the first nucleic acid sequence is placed in a functional relationship with the second nucleic acid sequence. For instance, a promoter is operably linked to a protein coding sequence if the promoter affects the transcription or expression of the protein coding sequence. Generally, operably linked DNA sequences are contiguous and, where necessary, join two protein-coding regions in the same reading frame. With respect to polypeptides, two polypeptide sequences may be operably linked by covalent linkage, such as through peptide bonds or disulfide bonds.

Recombinant: By “recombinant nucleic acid” herein is meant a nucleic acid that has a sequence that is not naturally occurring or has a sequence that is made by an artificial combination of two otherwise separated segments of sequence. This artificial combination is often accomplished by chemical synthesis or, more commonly, by the artificial manipulation of nucleic acids, e.g., by genetic engineering techniques, such as by the manipulation of at least one nucleic acid by a restriction enzyme, ligase, recombinase, and/or a polymerase. Once introduced into a host cell, a recombinant nucleic acid is replicated by the host cell, however, the recombinant nucleic acid once replicated in the cell remains a recombinant nucleic acid for purposes of this invention. By “recombinant protein” herein is meant a protein produced by a method employing a recombinant nucleic acid. As outlined above “recombinant nucleic acids” and “recombinant proteins” also are “isolated” as described above.

Non-naturally Occurring Plant: A non-naturally occurring plant is a plant that does not occur in nature without human intervention. Non-naturally occurring plants include transgenic plants and plants produced by non transgenic means such as plant breeding.

Transgenic plant: As used herein, this term refers to a plant or tree that contains recombinant genetic material not normally found in plants or trees of this type and which has been introduced into the plant in question (or into progenitors of the plant) by human manipulation. Thus, a plant that is grown from a plant cell into which recombinant DNA is introduced by transformation is a transgenic plant, as are all offspring of that plant that contain the introduced transgene (whether produced sexually or asexually). It is understood that the term transgenic plant encompasses the entire plant or tree and parts of the plant or tree, for instance grains, seeds, flowers, leaves, roots, fruit, pollen, stems etc.

Ortholog: Two nucleotide or amino acid sequences are orthologs of each other if they share a common ancestral sequence and diverged when a species carrying that ancestral sequence split into two species, sub-species, or cultivars. Orthologous sequences are also homologous sequences. Orthologous sequences hybridize to one another under high-stringency conditions. The term “polynucleotide”, “oligonucleotide”, or “nucleic acid” refers to a polymeric form of nucleotides of any length, either deoxyribonucleotides or ribonucleotides, or analogs thereof. The terms “polynucleotide” and “nucleotide” as used herein are used interchangeably. Polynucleotides may have any three-dimensional structure, and may perform any function, known or unknown. The following are non-limiting examples of polynucleotides: a gene or gene fragment, exons, introns, messenger RNA (mRNA), transfer RNA, ribosomal RNA, ribozymes, cDNA, recombinant polynucleotides, branched polynucleotides, plasmids, vectors, isolated DNA of any sequence, isolated RNA of any sequence, nucleic acid probes, and primers. A polynucleotide may comprise modified nucleotides, such as methylated nucleotides and nucleotide analogs. If present, modifications to the nucleotide structure may be imparted before or after assembly of the polymer. The sequence of nucleotides may be interrupted by non-nucleotide components. A polynucleotide may be further modified after polymerization, such as by conjugation with a labeling component. A “fragment” or “segment” of a nucleic acid is a small piece of that nucleic acid.

Taking into account these definitions, the present invention is directed to the finding that the sequence of the wheat AP1 promoter is the determining factor in distinguishing winter wheat from spring wheat in regards to the vernalization response. Winter wheats require several weeks at low temperature to flower. This process called vernalization is controlled mainly by the VRN1 gene. As detailed in the Example, using 6,190 gametes VRN1 was found to be completely linked to MADS-box genes AP1 and AGLG1 in a 0.03-cM interval flanked by genes Cysteine and Cytochrome B5. No additional genes were found between the last two genes in 324-kb of wheat sequence or in the colinear regions in rice and sorghum. The Example further shows that AP1 transcription is regulated by vernalization in both apices and leaves, and the progressive increase of AP1 transcription was consistent with the progressive effect of vernalization on flowering time. In addition, the Example indicates that vernalization is required for AP1 transcription in apices and leaves in winter wheat but not in spring wheat. No differences were detected between genotypes with different VRN1 alleles in the AP1 and AGLG1 coding regions, but three independent deletions were found in the promoter region of AP1.

In particular, all accessions with deletions that affect all, a portion or an adjacent region to the Car-G box region (SEQ ID NO:23) in the wheat AP1 promoter sequence have a spring growth habit. These results and the relatively later expression of AGLG1 during the flowering process demonstrate that AP1 is a better candidate for VRN1 than AGLG1.

The epistatic interactions between vernalization genes VRN1 and VRN2 suggested a model in which VRN2 would repress directly or indirectly the expression of AP1. As explained in detail below, a mutation in the Car-G section of the promoter region of AP1 would result in the lack of recognition of the repressor and in a dominant spring growth habit. The present invention is directed to this finding and its application to plant molecular biology and plant breeding.

The AP1 Promoter

The isolation and sequence analysis of the wheat AP1 promoter and the determination that it is the controlling factor in distinguishing winter wheat from spring wheat has broad applications in plant molecular biology and plant breeding.

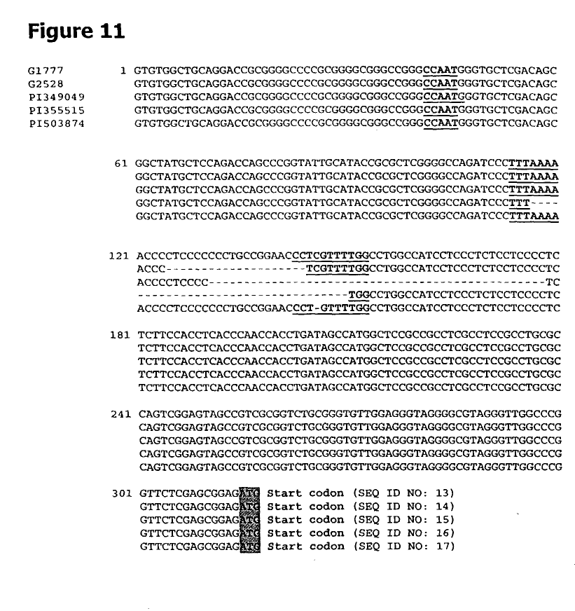

As a first embodiment, the present invention is directed to the AP1 promoter isolated from spring wheat. The winter wheat AP 1 promoter sequence, G3116, depicted in FIG. 9 (SEQ ID NO:12) contains a core Car-G box sequence CCTCGTFTTTTGG (SEQ ID NO:23). The spring wheat AP1 promoter sequence lacks all, a portion, or adjacent sequence to this core sequence. As such, the present invention is directed to a recombinant AP1 promoter sequence wherein the AP1 promoter sequence hybridizes to the nucleic acid molecule of SEQ ID NO:12 or the complement thereof under high stringency conditions wherein the AP1 promoter sequence lacks all or a portion of nucleotides −162 to −172 upstream the start codon of SEQ ID NO: 9, CCTCGTTTTGG (SEQ ID NO:23). Representative, but non-limiting spring wheat AP1 sequences are depicted in FIG. 11 as SEQ ID NO:14-17 wherein a portion of the CCTCGTTTTGG sequence is deleted or altered. An AP1 promoter sequence is said to lack all or a portion of the CCTCGTTTTGG sequence if 1, 2, 3, 4, 5, 6, 7, 8, 9, 10, 11 of the nucleotides of SEQ ID NO:23 are missing, absent, mutated, or subject to a sequence change or a deletion has been introduced in the sequence. The mutation may be an inversion, a reversion or other alteration in the sequence.

Vectors

The AP1 promoter sequences of the invention may be cloned into a suitable vector. Expression vectors are well known in the art and provide a means to transfer and express an exogenous nucleic acid molecule into a host cell. Thus, an expression vector contains, for example, transcription start and stop sites such as a TATA sequence and a poly-A signal sequence, as well as a translation start site such as a ribosome binding site and a stop codon, if not present in the coding sequence. A vector can be a cloning vector or an expression vector and provides a means to transfer an exogenous nucleic acid molecule into a host cell, which can be a prokaryotic or eukaryotic cell. Such vectors include plasmids, cosmids, phage vectors and viral vectors. Various vectors and methods for introducing such vectors into a cell are described, for example, by Sambrook et al. 1989.

The invention also provides an expression vector containing an AP1 promoter nucleic acid molecule operably linked to a protein coding sequence. For this construct, the AP1 promoter may be from any temperate grass but is preferably from a winter wheat or a spring wheat. In another format, the present invention is directed to a recombinant AP1 promoter sequence linked to an AP1 protein.

In the constructs of the invention, each component is operably linked to the next. For example, where the construct comprises the spring wheat AP1 promoter, and protein encoding sequence, preferably, the wheat AP1 protein, the AP1 promoter is operably linked to the 5′ end of the wheat AP1 protein encoding sequence or open reading frame.

The AP1 coding sequence may be from wheat or other AP1 protein coding sequences as defined herein. The protein coding sequence linked to the AP1 promoter may be an AP1 protein sequence or another heterologous protein. The heterologous proteins which find use in the invention include those that provide resistance to plant pests, faciliate translocation of nutrients, provide resistance to stresses typical of the summer: heat and dehydration, etc.

The constructs of the invention may be introduced into transgenic plants. A number of recombinant vectors suitable for stable transformation of plant cells or for the establishment of transgenic plants have been described including those described in Weissbach and Weissbach (1989), and Gelvin et al. (1990). Typically, plant transformation vectors include one or more open reading frames (ORFs) under the transcriptional control of 5′ and 3′ regulatory sequences and a dominant selectable marker with 5′ and 3′ regulatory sequences. Dominant selectable marker genes that allow for the ready selection of transformants include those encoding antibiotic resistance genes (e.g., resistance to hygromycin, kanamycin, bleomycin, G418, streptomycin or spectinomycin) and herbicide resistance genes (e.g, phosphinothricin acetyltransferase).

Standard molecular biology methods, such as the polymerase chain reaction, restriction enzyme digestion, and/or ligation may be employed to produce these constructs.

Transgenic Plants

Standard molecular biology methods and plant transformation techniques can be used to produce transgenic plants that produce plants having a recombinant AP1 promoter.

Introduction of the selected construct into plants is typically achieved using standard transformation techniques. The basic approach is to: (a) clone the construct into a transformation vector, which (b) is then introduced into plant cells by one of a number of techniques (e.g., electroporation, microparticle bombardment, Agrobacterium infection); (c) identify the transformed plant cells; (d) regenerate whole plants from the identified plant cells, and (d) select progeny plants containing the introduced construct.

Preferably all or part of the transformation vector will stably integrate into the genome of the plant cell. That part of the transformation vector which integrates into the plant cell and which contains the introduced recombinant sequence may be referred to as the recombinant expression cassette.

Selection of progeny plants containing the introduced transgene may be made based upon the detection of the recombinant AP1 promoter in transgenic plants, or upon enhanced resistance to a chemical agent (such as an antibiotic) as a result of the inclusion of a dominant selectable marker gene incorporated into the transformation vector.

Successful examples of the modification of plant characteristics by transformation with cloned nucleic acid sequences are replete in the technical and scientific literature. Selected examples, which serve to illustrate the knowledge in this field of technology include: U.S. Pat. No. 5,571,706 (“Plant Virus Resistance Gene and Methods”); U.S. Pat. No. 5,677,175 (“Plant Pathogen Induced Proteins”); U.S. Pat. No. 5,510,471 (“Chimeric Gene for the Transformation of Plants”); U.S. Pat. No. 5,750,386 (“Pathogen-Resistant Transgenic Plants”); U.S. Pat. No. 5,597,945 (“Plants Genetically Enhanced for Disease Resistance”); U.S. Pat. No. 5,589,615 (“Process for the Production of Transgenic Plants with Increased Nutritional Value Via the Expression of Modified 2S Storage Albumins”); U.S. Pat. No. 5,750,871 (“Transformation and Foreign Gene Expression in Brassica Species”); U.S. Pat. No. 5,268,526 (“Overexpression of Phytochrome in Transgenic Plants”); U.S. Pat. No. 5,780,708 (“Fertile Transgenic Corn Plants”); U.S. Pat. No. 5,538,880 (“Method for Preparing Fertile Transgenic Corn Plants”); U.S. Pat. No. 5,773,269 (“Fertile Transgenic Oat Plants”); U.S. Pat. No. 5,736,369 (“Method for Producing Transgenic Cereal Plants”); U.S. Pat. No. 5,610,049 (“Methods for Stable Transformation of Wheat”); U.S. Pat. No. 6,235,529 (“Compositions and Methods for Plant Transformation and Regeneration”) all of which are hereby incorporated by reference in their entirety. These examples include descriptions of transformation vector selection, transformation techniques and the construction of constructs designed to express an introduced transgene.

The transgene-expressing constructs of the present invention may be usefully expressed in a wide range of higher plants where an altered response to vernalization is useful. The invention is expected to be particularly applicable to monocotyledonous cereal plants including barley, wheat, rye, triticale, oat and forage grasses.

Methods for the transformation and regeneration of monocotyledonous plant cells are known, and the appropriate transformation technique will be determined by the practitioner. The choice of method will vary with the type of plant to be transformed; those skilled in the art will recognize the suitability of particular methods for given plant types. Suitable methods may include, but are not limited to: electroporation of plant protoplasts; liposome-mediated transformation; polyethylene glycol (PEG-mediated transformation); transformation using viruses; micro-injection of plant cells; micro-projectile bombardment of plant cells; vacuum infiltration; and Agrobacterium-mediated transformation. Typical procedures for transforming and regenerating plants are described in the patent documents listed above.

Following transformation, transformants are preferably selected using a dominant selectable marker. Typically, such a marker will confer antibiotic or herbicide resistance on the seedlings of transformed plants, and selection of transformants can be accomplished by exposing the seedlings to appropriate concentrations of the antibiotic or herbicide. After transformed plants are selected and grown the plant can be assayed for expression of recombinant proteins.

Uses of the Transgenic Plants of the Invention

The transgenic plants of the invention are useful in that they exhibit an altered response to vernalization. As defined herein, an altered response to vernalization means that the transgenic plant will respond differently to vernalization than a comparable non-transgenic plant. In one embodiment, a transgenic winter wheat expressing a recombinant spring wheat AP1 promoter operably coupled to an AP1 polypeptide sequence will exhibit an altered response to vernalization in that the recombinant AP1 protein will be expressed in the absence of vernalization and the plant will flower without the requirement of vernalization. In other words, a winter genotype would be transformed into a spring phenotype. Such expression contrasts to the expression of the endogenous (non-recombinant) AP1 protein in the transgenic plant, which requires vernalization for expression.

The protein coding sequence linked to the AP1 promoter may be also any heterologous protein. Heterologous proteins useful in the invention include proteins encoded by polynucleotides from any source, natural or synthetic. Suitable coding regions encode animal RNAs or polypeptides, as well as variants, fragments and derivatives thereof. The encoded products may be recovered for use outside the host plant cell (e.g., therapeutically active products) or they may alter the phenotype of the host plant cell (e.g., conferring disease resistance, the ability to survive or grow in the presence of particular substrates). Examples of such coding regions include polynucleotides derived from vertebrates, such as mammalian coding regions for RNAs (e.g., anti-sense RNAs, ribozymes, and chimeric RNAs having ribozyme structure and activity) or polypeptides (e.g., human polypeptide coding regions). Other coding regions useful in the inventive methods are derived from invertebrates (e.g., insects), plants (e.g., crop plants), and other life forms such as yeast, fungi and bacteria. The heterologous proteins which find particular use in the invention include those that provide resistance to plant pests, facilitate translocation of nutrients, provide resistance to stresses typical of the summer: heat and dehydration, etc. Such protein sequences are available in the literature and known to those of skill in the art. Representative proteins of interest are described and disclosed in Plant Biochemistry and Molecular Biology, 2nd Edition, Peter Lea and Richard C. Leegood editors; Plant Molecular Biology Second Edition; D. Grierson , S. N. Covey John Innes Institute, Norwich, UK Kluwer Academic Publishers, Dordrecht April 1991 and Biochemistry & Molecular Biology of Plants, edited, Bob B. Buchanan, Wilhelm Gruissem, and Russell L. Jones; John Wiley and Sons, Publishers, 2001, all of which are hereby incorporated by reference in their entirety.

In another embodiment, a transgenic plant will express any protein only after vernalization. This could be useful to avoid the expression of the transgene during the vegetative growth and to direct its expression to the flowering period of the plant.

Plants Produced by Plant Breeding

Results presented here demonstrated that the allelic variation at the AP1 gene is responsible for the allelic variation at the Vrn1 gene from wheat. Therefore allelic variation at the AP1 gene can be used as a molecular marker for the Vrn1 gene in marker assisted selection programs. Marker-assisted breeding is a procedure well known in the art as described in Hayward, et al.

These markers can be used to transfer different Vrn1 alleles into different germplasm by marker-assisted selection. They can also be used to determine the different haplotypes present in this region in the cultivated wheats and to establish a classification of the different haplotypes. This characterization will be useful to determine the adaptative value of the different haplotypes to different environments.

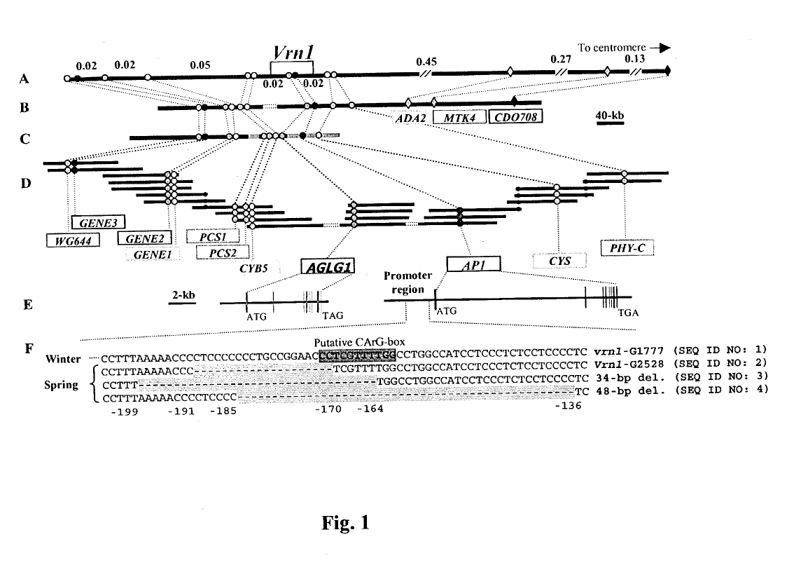

This invention relates to the use of allelic variation at any of the genes present in FIG. 1 as molecular markers for the Vrn1 gene.

This invention will be better understood by reference to the following non-limiting example.

### EXAMPLE

### Background

VRN1 and VRN2 (unrelated to the genes with similar names in Arabidopsis) are the main genes involved in the vernalization response in diploid wheat Triticum monococcum (1, 2). (Full citations for the references cited herein are provided before the claims.) However, most of the variation in the vernalization requirement in the economically important polyploid species of wheat is controlled by the VRN1 locus (2, 3). This gene is critical in polyploid wheats for their adaptation to autumn sowing and divides wheat varieties into the winter and spring market classes.

The VRN1 gene has been mapped in colinear regions of the long arm of chromosomes 5A (1, 3, 4), 5B (5, 6) and 5D (3). This region of wheat chromosome 5 is colinear with a region from rice chromosome 3 that includes the HD-6 QTL for heading date (7). However, it was recently demonstrated that VRN1 and HD-6 are different genes (8).

In spite of the progress made in the elucidation of the vernalization pathway in Arabidopsis, little progress has been made in the characterization of wheat vernalization genes. The two main genes involved in the vernalization pathway in Arabidopsis, FRI and FLC (9-11), have no clear homologues in the complete draft sequences of the rice genome (12). This may not be surprising considering that rice is a subtropical species that has no vernalization requirement. Since no clear orthologues of the Arabidopsis vernalization genes were found in rice or among the wheat or barley ESTs, a map based cloning project for the wheat VRN1 gene was initiated.

Chromosome walking in wheat is not a trivial exercise because of the large size of its genomes (5,600 Mb per haploid genome) and the abundance of repetitive elements (13, 14). To minimize the probability that these repetitive elements would stop the chromosome walking, simultaneous efforts were initiated in the orthologous regions in rice, sorghum, and wheat. The initial sequencing of rice, sorghum, and wheat BACs selected with RFLP marker WG644 (0.1 cM from VRN1) showed good microcolinearity among these genera (14-16). The low gene density observed in the wheat region and the large ratio of physical to genetic distances (14) suggested that large mapping populations and comparative physical maps would be necessary for a successful positional cloning of VRN1.

### Materials and Methods

Mapping Population

The high-density map was based on 3,095 F₂ plants from the cross between T. monococcum ssp. aegilopoides accessions G2528 (spring, VRN1) with G1777 (winter, vrn1). These two lines have the same dominant allele at the VRN2 locus and therefore, plants from this cross segregate only for VRN1 in a clear 3:1 ratio (1, 2).

Plants were grown in a greenhouse at 20-25° C. without vernalization and under long photoperiod (16-h light). Under these conditions, winter plants flowered one to two months later than spring plants. F₂ plants are analyzed for molecular markers flanking VRN1, and progeny tests are performed for plants showing recombination between these markers. The 20-25 individual F₃ plants from each progeny test were characterized with molecular markers flanking the crossover to confirm that the observed segregation in growth habit was determined by variation at the VRN1 locus. G2528 and G1777 were included as controls in each progeny test.

For studies to confirm that the CarG-box is the critical site for the recognition of the vernalization signal the following steps were taken. PI503874 (spring wheat with a single bp deletion in the CarG box SEQ 17) was crossed G3116 (winter wheat). An F1 plant was produced with spring flowering habits (no vernalization requirement) indicative of a dominant spring growth habit. F1 plants were self-pollinated to produce 144 F2 seeds. The 144 F2 seeds were planted in cones and grown without vernalization in the green house under long day conditions. DNA was extracted from each of the plants and the promoter region was amplified by PCR. The amplified PCR fragment was digested with a restriction enzyme that cut only the sequence without the one base pair deletion.

Procedures for genomic DNA extraction, Southern blots, and hybridizations were described before (17). The first 500 F₂ plants were screened with flanking RFLP markers CDO708 and WG644 (1), which were later replaced by closer PCR markers to screen the complete mapping population. Additional markers were developed for the eight genes present between the PCR markers as detailed below.

Molecular Markers

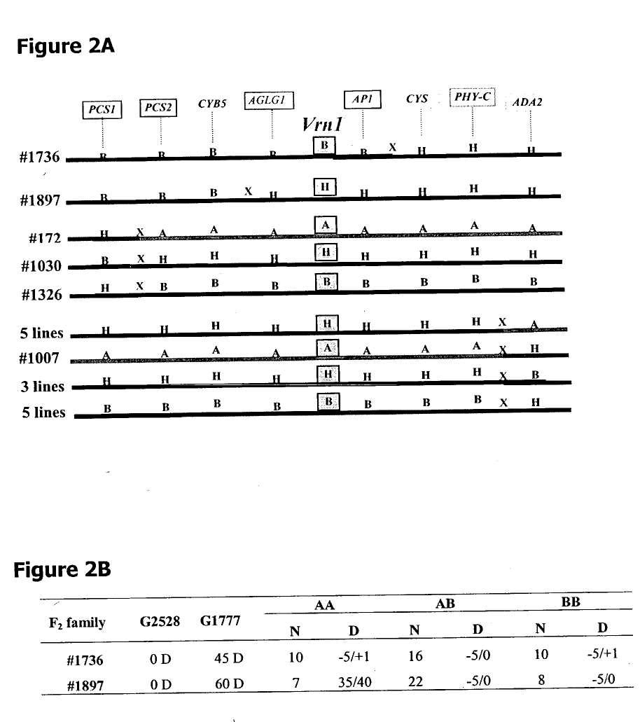

Molecular markers were developed for the high-density map of the Triticum monococcum Vrn1 vernalization gene as depicted in FIGS. 1-2. The information is organized by the order of genes in FIG. 1. All primers are 5′ to 3′. The Cleavage Amplification Polymorphic Sequence (CAPS) markers show PCR products digested with the polymorphic restriction enzyme. The PCR products are detectable by gel electrophoresis.

a) RFLP Marker WG644

Sequence of the WG644 showed that this RFLP marker was part of GENE4 (putative ABC transporter gene) present in T. monococcum BAC clone 115G01 (AF459639). This wheat RFLP marker was polymorphic between G1777 and G2528 with restriction enzyme DraI.

b) GENE1 (putative Mitochondrial Carrier Protein,
AF459639)
Primers

| | | |
|---|---|---|
| GENE1-F | CCAGCGTATGATTTGGAGGT | (SEQ ID NO: 24) |
| GENE1-R | TTGGCATTATTGGACCATCA | (SEQ ID NO: 25) |

Sequence of the G1777 (AY244503) and G2528 (AY244504) alleles showed a polymorphic Taq I restriction site. This polymorphism was used to develop a CAPS marker.

c) PCS1 (Phytochelatin synthetase)
Primers

| | | |
|---|---|---|
| PCS1-F | CTGACCTGGGGCCTTGAGAG | (SEQ ID NO: 26) |
| PCS1-R | CTTCGCATCAGCAGCTCTAT | (SEQ ID NO: 27) |

These primers amplified a 507 bp region of the Phytochelatin Synthetase pseudogene (AY188332). This wheat RFLP marker was polymorphic between G1777 and G2528 with restriction enzyme DraI.

d) PCS2 (Phytochelatin synthetase)
Primers

| | | |
|---|---|---|
| PCS2-F | CCATGGATAATCATCGGGAG | (SEQ ID NO:28) |
| PCS2-R | GTCACCATCACCAACTTCAA | (SEQ ID NO:29) |

Primers were used to amplify a region of the Phytochelatin Synthetase 2 gene (Exons 3-4) from barley variety Morex (AY244504). This RFLP marker was polymorphic between G1777 and G2528 with restriction enzyme Eco RI.

e) CYB5 (Cytochrome B5)
Primers

| | | |
|---|---|---|
| CYB5-F | GACTGCGTATTTGGACGACC | (SEQ ID NO:30) |
| CYB5-R | CCACGGCTGATATCCCGACTG | (SEQ ID NO:31) |

These primers amplify a 373-bp region of Cytochrome B5 gene (Exons 2-3) from T. monococcum BAC clone 609E06 (AY188332). This RFLP marker was polymorphic between G1777 and G2528 with restriction enzyme Eco RI.

f) AGLG1 (MADS-box)
Primers

| | | |
|---|---|---|
| AGLG1-F | GACCCTCGAGAGGTACCAG | (SEQ ID NO:32) |
| AGLG1-R | CATCTACACTACGATCTAGC | (SEQ ID NO:33) |

These primers amplified exon2 and intron 2 of AGLG1 from T. monococcum BAC 719C13. Sequence of the G1777 (YA244506) and G2528 (YA244507) alleles showed two polymorphic Dpn II restriction sites. A cDNA from this gene (BE430753) was also mapped by RFLP using Eco RI to delimit the region of the crossover between AGLG1 and CYB5.

g) PHY-C (Phytochrome-C)
Primers based on barley EST BE060096

| | | |
|---|---|---|
| PHY-C-F | GAAAATGTCTGAACAAGCTGCT | (SEQ ID NO:34) |
| PHY-C-R | TCTAGATGAGCAATCTGCAT | (SEQ ID NO:35) |

These primers were used to amplify a 750-bp product form G1777 (AY244514) that was used as an RFLP probe to map a Hha I polymorphism.

h) ADA2
(Transcriptional Adaptor, Zea mays AJ430205)
Primers based on T. aestivum EST BJ309328

| | | |
|---|---|---|
| ADA2-F | GAAGATGCACTTGGAGAAGG | (SEQ ID NO: 36) |
| ADA2-R | GTCTCTTTGCATTGTACCCA | (SEQ ID NO: 37) |

These primers were used to amplify a 700-bp product from G1777 (AY244515) that was used as an RFLP probe to map an Rsa I polymorphism.

i) MTK4 (Tousled-like Kinase, AC091811)
Primers

| | | |
|---|---|---|
| MTK4_F | GGTAAAAGATGAGCAAGGAG | (SEQ ID NO: 38) |
| MTK4-R | TCTATCTATGGTGAACTCTTACTTC | (SEQ ID NO: 39) |

Sequencing of G1777 (AY244512) and G2528 (AY244513) alleles with these primers showed a polymorphic Dpn II restriction site that was used to develop a CAPS marker.

j) CDO708 (putative RNA-binding protein, AC091811) The CDO708 clone was sequenced with primer M13 Forward. This sequence had a high homology to putative RNA-binding protein AAL58954.1 from rice BAC AC091811. This clone was used as an RFLP probe to map a Hha I polymorphism between G1777 and G2528.

Contig Construction and BAC Sequencing

High-density filters for the BAC libraries from T. monococcum accession DV92 (18), Oryza sativa var. Nipponbare (19), and Sorghum bicolor (20) were screened with segments from the different genes indicated in FIG. 1. Contigs were assembled using Hind III fingerprinting and confirmed by hybridization of BAC ends obtained by plasmid rescue, inverse PCR (20) or BAC sequencing. Restriction maps using single and double digestions with eight-cutter restriction enzymes, pulse field electrophoresis, and hybridization of the Southern blots with different genes, were used to order genes within the BACs, to select the fragment sequenced from the sorghum BAC, and to confirm the assembly results from the BAC sequencing. Shotgun libraries for BAC sequencing were constructed as described before (15). Complete T. monococcum BACs 609E06, 719C13, and 231A16 and a 24-kb fragment from sorghum BAC 17E12 were sequenced. Genes were identified by a combination of comparative genomic analysis, BLAST searches and gene-finding programs (15).

Phylogenetic Analysis

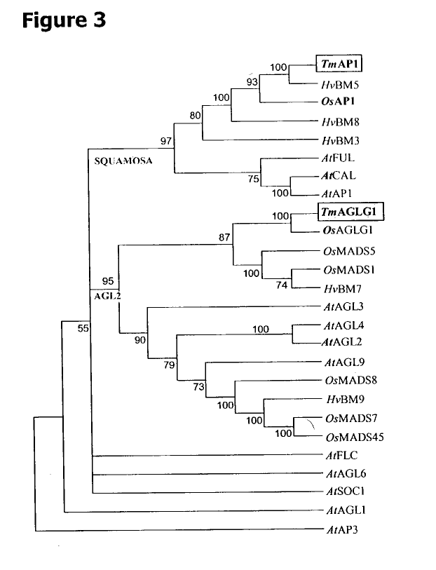

A phylogenetic study was performed using the two wheat MADS-box genes found in this study and 24 additional MADS-box genes (FIG. 3). Phylogenetic trees were generated from the ClustalW sequence alignments of the complete proteins using multiple distance- and parsimony- based methods available in the MEGA2.1 computer software package (21). Distances between each pair of proteins were calculated and a tree was constructed using the Neighbor-Joining algorithm. The consensus tree and the confidence values for the nodes were calculated using 1000 bootstraps (MEGA2.1). GenBank accessions used in the study were SQUAMOSA: HvBM5 (CAB97352.1), OsAP1 (AAM34398.1), HvBM8 (CAB97354.1), HvBM3 (CAB97351.1), AtFUL (Q38876), AtCAL (NP_{—}564243.1), AtAP1 (CM78909.1); AGL2: OsAGLE21 (AAM34397.1), OsMADS5 (AAB71434.1), OsMADS1 (AAA66187.1), HvBM7 (CAB97353.1), AtAGL3 (AAB38975.1), AtAGL4 (AAA32734.1), AtAGL2 (AAA32732.1), AtAGL9 (AAB67832.1), OsMADS8 (AAC49817.1), HvBM9 (CAB97355.1), OsMADS7 (AAC49816.1), OsMADS45 (AAB50180.1); OTHER: AtFLC (MD21249.1), AtAGL6 (AAA79328.1), AtSOC1 (AAG16297.1), AtAGL1 (AAA32730.1), AtAP3 (AAA32740.1).

RT-PCR and Quantitative PCR

RNA from leaves, undifferentiated apices, and young spikes was extracted using the TRIZOL method (INVITROGEN). RT-PCR procedures were performed as described before (22). Quantitative PCR experiments were performed in an AB17700 using three TaqMan® systems for T. monococcum AP1 and for ACTIN and UBIQUTIN as endogenous controls. RT-PCR and Quantitative PCR experiments for Triticum monococcum AP1 gene. All probes and primers are indicated in 5′ to 3′ orientation. RT-PCR reactions were performed using Superscript II (Invitrogen®) and primed with oligo(dT)₁₂₋₁₈.

The 2^{-ΔΔC}_{T} method (23) was used to normalize and calibrate the C_{T} values of wheat AP1 relative to the endogenous controls. For the vernalization time course, RNA was extracted from the youngest fully expanded leaf of five winter T. monococcum plants (1 month old) immediately before moving the plants into the cold room, and then after 2, 4, and 6 weeks of vernalization (4° C.). The last sample was collected two weeks after moving plants to the greenhouse (20° C.). Plants kept in the greenhouse were sampled as controls at each time point simultaneously with the plants from the cold room (5 plants per time point).

a) RT-PCR

RT-PCR reactions were performed using superscript II (invitrogen®) and primed with oligol (^{dT}) 12-18. AM probes and primers are indicated in the 5′ to 3′ orientation.

AP1

The Left primer, Exon 3 was GGAAACTGGTGTCACGAATA (SEQ ID NO:40). The Right primer 5′ UTR was CAAGGGGTCAGGCGTGCTAG (SEQ ID NO:41)

The cDNA product: 571-bp and the Genomic DNA product was 1262-bp. The AP1-specificity of the 5′UTR primer was confirmed by sequencing the PCR amplification products. Hybridization of the PCR product with Southern blots of T. monococcum indicated that AP1 was a single copy gene in T. monococcum.

AGLG1

The Left primer Exon 2 was GAGGATTTGGCTCCACTGAG (SEQ ID NO:42). The Right primer. Exon 7 outside K-box was TCTAGGGCCTGGAAGAAGTG (SEQ ID NO:43).

The cDNA product was 302-bp in length. The Genomic DNA product was 901-bp.

The AGLG1-specificity of these primers was confirmed by sequencing the PCR amplification products. Hybridization of the PCR product with Southern blots of T. monococcum indicated that AGLG1 was a single copy gene in T. monococcum.

ACTIN

The Left primer, Exon 3 was ATGTGGATATCAGGAAGGA (SEQ ID NO:44). The Right primer, Exon 3 was CTCATACGGTCAGCAATAC (SEQ ID NO:45)

The cDNA product: 85-bp

b) Quantitative PCR

Tests for amplification efficiency were performed. Six 2-fold dilutions were tested in triplicate; 1:1, 1:2, 1:4, 1:8, 1:16, 1:32. Standard curves were plotted with ng RNA on the X-axis and ΔC_{T} on the y-axis. The slope and the differences in slopes with the 18S standard curve were determined. The criteria for passed test was set as the differences of slopes being <0.1. The calculation of the efficiency based on the slope was also plotted.

ACTIN TaqMan System

The Left primer was: ATGGAAGCTGCTGGAATCCAT (SEQ ID NO:46). The Probe (reverse orientation) was CCTTCCTGATATCCACATCACACTTCATGATAGAGT (SEQ ID NO:47)

The Right primer is: CCTTGCTCATACGGTCAGCAATAC (SEQ ID NO:48)

The sequence of Actin exon 3 is:

| | |
|---|---|
| GAGAAGAGCTATGAGCTGCCTGATGGGCAGGTGA | (SEQ ID NO: 49) |
| TCACCATTGGGGCAGAGAGGTTCCGTTGCCCTGA | |
| GGTCCTTTTCCAGCCATCTTTCATTGGTATGGAA | |
| GCTGCTGGAATCCATGAGACCACCTACAACTCTA | |
| TCATGAAGTGTGATGTGGATATCAGGAAGGATCT | |
| GTATGGTAACATCGTGCTCAGTGGTGGCTCAACT | |
| ATGTTCCCGGGTATTGCTGACCGTATGAGCAAGG | |
| AGATCACTGCCCTTGCACCAAGCAGCATGAAGAT | |
| CAAGGTGGTGGCACCGCCTGAGAGGAAGTACAGT | |
| GTCTGGATTGGAGGGTCGATTCTTGCCTCCCTTA | |
| GTACCTTCCAACAG | |

The differences of the slopes with 18S was determined to be 0.0352. The actin system passed the efficiency test with an efficiency of 99.1.

AP1 TaqMan System

The Left primer was: AACTCAGCCTCAAACCAGCTCTT (SEQ ID NO:50). The Probe (reverse orientation) was CATGCTGAGGGATGCTCCCCCTG (SEQ ID NO:51). The Right primer was CTGGATGAATGCTGGTATTTGC (SEQ ID NO:52).

| | |
|---|---|
| The AP1 T. monococcum sequence is: | |
| CTCGTGGAGAAGCAGAAGGCCCATGCGGCGCAGC | (SEQ ID NO: 53) |
| AAGATCAAACTCAGCCTCAAACCAGCTCTTCTTC | |
| TTCTTCCTTCATGCTGAGGGATGCTCCCCCTGCC | |
| GCAAATACCAGCATTCATCCAGCGGCGGCAGGCG | |
| AGAGGGCAGAGGATGCGGCAGTGCAGCCGCAGGC | |
| CCCACCCCGGACGGGGCTTCCACCGTGGATGGTG | |
| AGCCACATCAACGGGTGA | |

The differences of the slopes with 18S was determined to be 0.0056. The actin system passed the efficiency test with an efficiency of 96.3.

UBIQUITIN TaqMan System

The Left primer was: ATGCAGATCTTTGTGAAGACCCTTAC (SEQ ID NO:54). The Probe was: CAAGACCATCACTCTGGAGGTTGAGAGCTC (SEQ ID NO:55). The Right primer GTCCTGGATCTTGGCCTTGA (SEQ ID NO:56)

The sequence of Ubiquitin is:

| | |
|---|---|
| ATGCAGATCTTTGTGAAGACCCTTACTGGCAAGA | (SEQ ID NO: 57) |
| CCATCACTCTGGAGGTTGAGAGCTCAGACACCAT | |
| CGACAATGTCAAGGCCAAGATCCAGGACAAGGAG | |
| GGCATCCCCCCGGACCAGCAGCGCCTCATCTTCG | |
| CAGGAAAGCAGCTGGAGGATGGCCGCACTCTTGC | |
| TGACTACAACATCCAGAAGGAGTCCACTCTTCAC | |
| CTTGTCCTGCGTCTTCGTGGCGGT | |

The differences of the slopes with 18S was determined to be 0.0292. The actin system passed the efficiency test with an efficiency of 99.4.

Additional Deletions in the Promoter Region of AP1

PCR primers for the promoter region flanking the 20-bp deletion present in the spring genotype G2528 were used to screen a collection of 65 accessions of cultivated T. monococcum ssp. monococcum. None of the winter accessions showed deletions in this region. Among the accessions with spring growth habit, three (PI-349049, PI326317, and PI 418582) showed a 34-bp deletion, one (PI-355515) showed a 48-bp deletion and one showed a 1 nucleotide change in the CarG box (FIG. 11).

The Primers used to screen the T. monococcum collection were:

AP1_ProDel_F1: ACAGCGGCTATGCTCCAG (SEQ ID NO:58) and

AP1_ProDel_R1: TATCAGGTGGTTGGGTGAGG (SEQ ID NO:59). The expected size without deletion is 152 bp

Deletions are illustrated in FIG. 11. Accessions carrying the new deletions can be crossed with winter T. monococcum ssp. boeticum G3116 to determine the linkage between these deletions and growth habit. A detailed sequence analysis of the allelic variation at the Vrn1 and Vrn2 loci in this collection can be prepared by procedures available to those of skill in the art.

The CarG-box was confirmed as a critical site for the recognition of the vernalization signal. The sequence of spring Triticum monococcum accession number PI503874 showed the presence of a 1-pb deletion in the CarG-box of the promoter of the AP1 gene. The normal CarG box is CCCTCGTTTTGG and the sequence in PI503874 was CCCT-GTTTTGG. The sequence of the promoter region T. monococcum P1503874 is provided in FIG. 11 (SEQ ID No:17). This one base pair deletion was the only observed difference with the promoters from other T. monococcum accessions with winter growth habit. Thirty-four plants were winter and 110 were spring. All the spring plants were homozygous or heterozygous for the presence of the one-base pair deletion, whereas the 34 winter plants were all homozygous for the absence of the one-bp deletion. This confirms complete linkage between the 1-bp deletion and the spring growth habit.

Marker Development

In the initial genetic map (1) the VRN1 gene was flanked in the distal side by WG644 and in the proximal side by CDO708. These markers were used as anchor points to the rice genome sequence to find additional markers.

Distal region: WG644 was previously used to identify rice BAC 36I5 that included GENE1 at its proximal end (15). BLASTN searches of the different rice genome projects using GENE1 and the end sequence of BAC 36I5 (AY013245) identified the connected contig CLO13482.168 (12). Two additional genes, Phytochelatin synthetase (PCS, Zea mays AAF24189.1) and Cytochrome B5 (CYB5, NP_{—}173958.1), were discovered and annotated in this new contig. These genes were mapped in wheat by RFLP (FIGS. 1 and 2A-2B).

Proximal region: RFLP marker CDO708 was mapped 0.9 cM proximal to VRN1 in the T. monococcum map. The sequence of this clone showed a high homology to a putative RNA-binding protein (AAL58954.1) located in rice BAC AC091811. The end of this rice BAC also included gene MTK4 (putative protein kinase tousled, AAL58952.1) that was converted into a PCR marker and was mapped in wheat (FIG. 1). Rice BAC sequence AC091811 was then connected through contigs CL039395.93, CL039395.83, and CL018222.111.1 (12) to rice BAC sequences AC092556 and AF377947. BAC sequence AC092556 included a Transcriptional Adaptorgene (ADA2, AJ430205) that was mapped in T. monococcum 0.5 cM from the VRN1 gene (FIG. 1). The last rice BAC sequence AF377947 included genes Phytochrome-C(PHY-C, AAM34402.1), Cysteine proteinase (CYS, AAM34401.1) and MADS-box genes AAM34398.1 and AAM34397.1, designated hereafter AP1 and AGLG1 (AGL-like gene from Grasses). The rice proximal region included 318-kb of contiguous sequence.

High-Density Genetic Maps of the VRN1 Region

The PCR markers developed for GENE1 and MTK4 were used to screen 6,190 chromosomes for recombination. Fifty-one recombinant events were detected, and those plants were further characterized using molecular markers for all the genes present between these two markers in rice (FIG. 1 and FIGS. 2A-2B). Progeny tests were performed for 30 of the 51 F₂ plants, to determine the VRN1 genotype of the parental F₂ plants. Based on the mapping information, the VRN1 locus was completely linked to AP1 and AGLG1.

On the proximal side, genes PHY-C and CYS flanked the VRN1 locus. The last two genes were completely linked to each other and separated from VRN1 by a single crossover (FIG. 1, FIGS. 2A-2B). On the distal side, the CYB5 gene was also separated from VRN1 by a single crossover. Comparison of genotypic and phenotypic data from all the F₃ plants used in the 30 progeny tests confirmed that the observed segregation in growth habit was determined by variation at the VRN1 locus. Unvernalized plants homozygous for the G1777 AP1 allele flowered 1-2 months later than G2528 whereas the other plants flowered only one week before or after the G2528 control. These results confirmed the simple Mendelian segregation for vernalization requirement in this cross (1).

Physical Maps

Distal contig: Genes CYB5 and GENE1 were used to screen the BAC libraries from T. monococcum, rice and sorghum. Triticum monococcum BAC clone 609E6 selected with the CYB5 gene was connected to previously sequenced 116F2 (AF459639) by four BACs (FIG. 1). The PCS gene hybridized with two fragments from BAC 609E06 (PCS1 and PCS2, FIG. 1) whereas only one PCS copy was found in the colinear region in rice. No single copy probes were found in BACs 609E6 or in the unique Hind III fragments from the most proximal BAC 393O11 to continue the chromosome walking towards the proximal region.

Proximal conting: Screening of the T. monococcum BAC library with PHY-C, CYS, AP1, and AGLG1 yielded twelve BACs organized in two contigs. The largest contig included eight BACs that hybridized with genes PHY-C, CYS, and AP1. The four additional BACs hybridized only with the AGLG1 gene (FIG. 1). The location of the AGLG1 contig within the physical map was determined by the complete linkage between the single copy genes AGLG1 and AP1 and the proximal location of AGLG1 relative to single copy gene CYB5. No additional single copy probes were found to close the gaps flanking the AGLG1 contig.

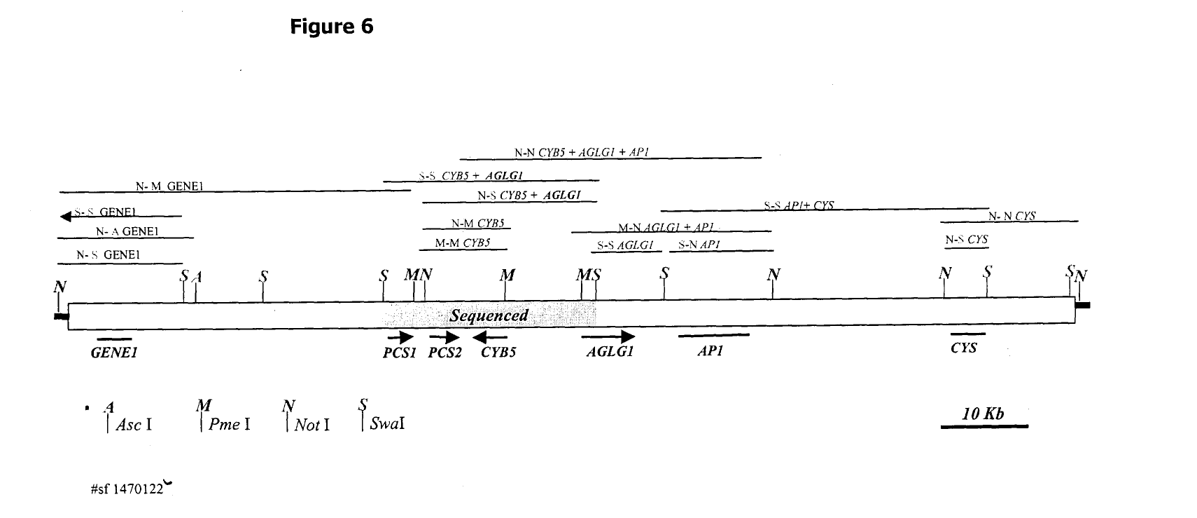

The proximal gap between AGLG1 and AP1 was covered by the current rice sequence. However, the distal gap between CYB5 and AGLG1 was also present in the different rice genome sequencing projects. The screening of the Nipponbare BAC libraries with probe CYB5 failed to extend the rice region because of the presence of a gap in the current rice physical maps. Fortunately, sorghum BAC 17E12 included GENE1, PCS1, PC52, and CYB5 genes from the distal contig, and AGLG1, AP1, and the Cys genes from the proximal contig, bridging the gap present in the rice and wheat contigs (FIG. 1). A restriction map of sorghum BAC 17E12 (FIG. 6) indicated that the sorghum genes were in the same order as previously found in rice and wheat and that a 24-kb Swa I-Swa I restriction fragment spanned the region of the rice and wheat gap between CYB5 and AGLG1.

Sequence Analysis

Annotated sequences from the three T. monococcum BACs (AY188331, AY188332, AY188333) and the partial sequence of the sorghum BAC 17E12 (AY188330) were deposited in GenBank. Including BACs 115G01 and 116F02 (AF459639) a total of 550-kb were sequenced. Multiple retrotransposons organized in up to four layers of nested elements were the most abundant features, similar to wheat regions analyzed before (13, 14). Retrotransposons and other repetitive elements accounted for 78.4% of the annotated sequence whereas genes represented only 8.5% of the total. The genes detected in this sequence were in the same order as the ones present in the corresponding regions in rice and sorghum, indicating an almost perfect microcolinearity. The only exception was the duplication of the PCS gene in sorghum and wheat relative to the presence of a single PCS gene in the colinear rice region (FIG. 1).

No additional genes were found in the rice sequence between the two MADS-box genes corresponding to one of the two gaps in the wheat physical map. These two genes were also adjacent in sorghum (FIG. 1, and FIG. 6). Similarly, no new genes were found between CYB5 and AGLG1 in the sequence of the 24-kb Swa I-Swa I restriction fragment from sorghum BAC 17E12 (AY188330) that covered the other gap in the wheat physical map. The four genes present in the sorghum sequence were in the same order and orientation as previously found in rice and wheat (FIG. 6). FIG. 6 shows: 1) No additional genes were detected between CYB5 and AGLG1. 2) No similar sequences were detected between the intergenic regions in sorghum and the colinear sequences in rice or wheat. 3) Genes in sorghum were in the same order as in wheat and rice.

The absence of new genes in the colinear regions of rice and sorghum, together with the excellent microcolinearity detected in this region, suggested that it would be unlikely to find additional genes in the current gaps of the wheat physical map. This assumption was also supported by the absence of any new gene in the 324-kb of wheat sequence flanking these gaps. The presence of almost uninterrupted series of nested retrotransposons flanking the gaps also explained the failure to find single copy probes to close the two gaps.

Classification of the Two MADS-Box Genes

The AP1 and AGLG1 proteins have MADS-box and K domains characteristic of homeotic genes involved in the flowering process and similar exon structure (FIG. 1, FIG. 6) (24). The consensus tree for 26 plant MADS-box proteins (FIG. 3) showed that the closest proteins to wheat AP1 and AGLG1 belonged to the SQUAMOSA (bootstrap 97) and AGL2 groups (bootstrap 95) respectively.

The closest Arabidopsis MADS-box proteins to wheat AP1 were the proteins coded by the three related meristem identity genes AP1, CAL and FUL (FIG. 3). Two separate clusters were observed in the SQUAMOSA group dividing the Arabidopsis and grass proteins. A similar separation between the monocot and dicot proteins was found in more detailed studies of this group (25). The AP1 protein from T. monococcum was 98.4% similar to previously described T. aestivum WAP1, formerly TaMADS#11 (26, 27), and 96.0% similar to barley BM5 (28). These two putative orthologous genes were described in papers characterizing the MADS-box family in wheat and barley, but were not mapped or associated with the VRN1 gene.

The wheat AGLG1 protein was clustered with members of the AGL2 subgroup and was closely related with the rice AGLG1 orthologue and with rice OsMADS5, OsMADS1 and barley BM7 proteins (bootstrap 87, FIG. 3).

Expression Profiles

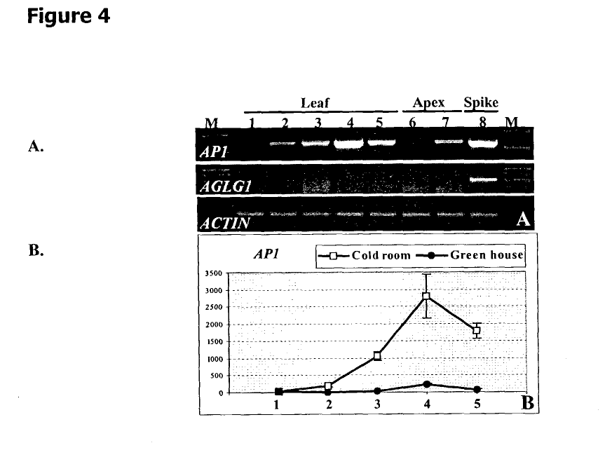

No AP1 transcripts were detected in apices from unvernalized plants of T. monococcum with strong winter growth habit (G3116) even after ten months in the greenhouse under long day conditions. However, AP1 transcription was detected in the apices of plants from the same genotype after six weeks of vernalization (FIG. 4A, lanes 6 and 7). The same result was obtained in three independent experiments. These apices were morphologically at vegetative stage zero according to the developmental scale of Gardner et al. (29). In T. monococcum accessions with spring growth habit, AP1 transcripts were observed in the apices without the need of previous vernalization.

Developmental Stage of the Apexes Used in the RT-PCR Experiment

After six weeks of vernalization the shoot apexes did not show any morphological sign of differentiation from the vegetative shoot apex stage as observed before vernalization. An apex from winter T. monococcum accession G3116 after six weeks of vernalization was visualized. The results showed that the expression of Ap1 in the apices precedes the differentiation of the apex.

Transcripts of AP1 were also detected in the leaves, as previously reported for WAP1 (26) and BMS(28). A quantitative PCR experiment using the endogenous controls ACTIN and UBIQUITIN demonstrated that transcription of AP1 in the leaves of the winter genotypes was also regulated by vernalization.

Effect of vernalization on ACTIN and UBIQUITIN transcription levels Average threshold cycle (C_{T})

Effect of vernalization on ACTIN and UBIQUITIN transcription levels
Average threshold cycle (C_{T})

| | n | Cold room C_{T} | n | Greenhouse C_{T} | ANOVA |
|---|---|---|---|---|---|
| ACTIN | 15 | 17.4 | 30 | 17.5 | P = 0.99 |
| UBIQUITIN | 14 | 15.1 | 29 | 15.3 | P = 0.55 |

No significant differences were detected between plants in the greenhouse and plants in the cold room in the C_{T} values of ACTIN and UBIQUITIN. The abundance of AP1 transcripts started to increase after the first two weeks of vernalization and continue increasing during the four additional weeks of the vernalization process (FIG. 4B).

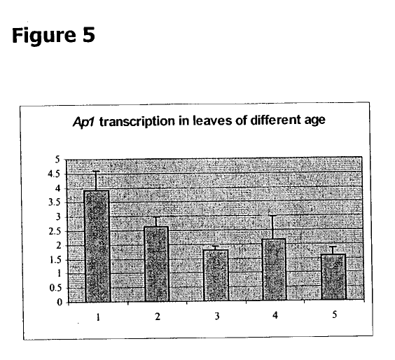

The AP1 transcription levels relative to ubiquitin are presented in FIG. 5. Samples were extracted from the emerging and first fully expanded leaves of Triticum monococcum G3116 (winter growth habit) 1) Before vernalization, 2) 2 weeks in the cold room, 3) 4 weeks in the cold room, 4) 6 weeks in the cold room, 5) two weeks after the vernalized plants were returned to the greenhouse. Units are linearized values using the 2^{(-ΔΔCT)} method, where CT is the threshold cycle. The results show that AP1 transcripts were also present in the leaves from vernalized plants two weeks after their transfer to the greenhouse.

Control plants kept in the greenhouse showed very low level of AP1 transcription during the eight weeks of the vernalization experiment (FIG. 4B). In the genotypes with a spring growth habit, AP1 transcripts were observed in the leaves of unvernalized plants that were initiating the transition to flowering.

AP1 transcription levels in leaves of different ages are shown in FIG. 5. One-month-old G3116 plants were vernalized for six weeks and then transferred to the greenhouse for two weeks under long day conditions. RNA was extracted from each of the five green leaves from the main stem and one secondary tiller from two plants. In FIG. 5, number 1 indicates the youngest leaf (not fully emerged from the sheath) and number 5 the oldest green leaf. Bars represent standard errors of the means. Ubiquitin was used as an internal control. Units are linearized values using the 2^{(-ΔΔCT)} method, where CT is the threshold cycle. The results show that AP1 transcripts were detected in young and old green leaves.

A relatively high level of expression of Ap1 was observed in all the leaves. Average C_{T} values for Ap1 (24.1) were only two cycles higher than for Ubiquitin (22.0). This result confirmed that Ap1 induction by vernalization was not restricted to the youngest leaves. Marginally significant differences (ANOVA, P=0.05) were observed between leaves of different ages, with the highest value for leaf 1

AGLG1 transcripts were detected only in young spikes (FIG. 4A, lane 8), but were not observed in the same cDNA samples from apices after six weeks of vernalization where the AP1 transcripts were already present (FIG. 4A). This indicates that AGLG1 transcription is initiated later than AP1. Transcripts from AGLG1 were not detected in the leaves (FIG. 4A).

The expression results together with the known role of the AP1 homologues in Arabidopsis as meristem identity genes, suggested that AP1 was a better candidate gene for VRN1 than AGLG1.

Allelic Variation

Four AP1 genes were sequenced from T. monococcum accessions G1777, G3116, and DV92 carrying the vrn1 allele and G2528 carrying the Vrn1 allele (1, 2). The nucleotide sequences for G2528 and DV92 are presented in FIG. 7. The predicted proteins from DV92 and G2528 were identical and differed from the predicted proteins from G3116 and G1777 by a single amino acid (FIG. 8).

Analysis of the 1024-bp region upstream from the AP1 start-codon and up to the insertion point of a large repetitive element (AY188331) showed the presence of five polymorphic sites. Two of them differentiated G2528 from the three accessions carrying the vrn1 allele for winter growth habit. One was a one bp insertion located 728-bp upstream from the start codon and the other one was a 20-bp deletion located 176-bp upstream from the start codon (FIG. 1, FIGS. 9A-9B, 11). No difference were detected in the first 600-bp of the AP1 3′ region between the vrn1 and Vrn1 alleles.

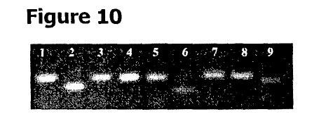

A PCR screening of a collection of cultivated T. monococcum accessions with primers flanking the 20-bp deletion region revealed the presence of deletions of different sizes in agarose gels (FIG. 10). Sequencing of these lines showed the presence of two new deletions that overlapped with the 20-bp deletion from G2528. These new deletions included a putative MADS-box protein-binding site adjacent to the 20-bp deletion (FIG. 1, FIG. 11).

No DNA differences were detected between accessions DV92 (vrn1) and G2528 (Vrn1) in the coding region, or the 5′ (365-bp) and 3′ (583-bp) untranslated regions of the AGLG1 gene.

### Discussion

Genetic and Physical Maps of the VRN1 Region

Only eight genes were found in the 556-kb of sequence from the T. monococcum VRN1 region, resulting in an estimated gene density of one gene per 70-kb. The low gene density observed in this region was paralleled by a high ratio between physical and genetic distances. Excluding the two gaps in the physical map, a minimum ratio of 6,250-kb cM⁻¹ was estimated for the region between WG644 and PHY-C This value is two times larger than the average genome-wide estimate of 3,000-kb cM⁻¹ (30) and four times larger than the 1,400-kb cM⁻¹ reported for the telomeric region of chromosome 1A (31). Previous cytogenetic studies demonstrated that recombination in the wheat chromosomes decrease exponentially with distance from the telomere (32, 33), predicting an increase of the ratio between physical and genetic distance in the same direction. The region studied here is located between the breakpoints in deletion lines 5AL-6 (FL 0.68) and 5AL-17 (FL 0.78), in a more proximal location than regions used before to estimate ratios between physical and genetic distances in wheat. This result suggests that positional cloning projects in the proximal regions of wheat will be difficult and would greatly benefit from the use of the rice genomic sequence to jump over large blocks of repetitive elements.

In spite of the low recombination rate found in this region, the large number of evaluated gametes was sufficient to find crossovers between most of the genes or at least between pairs of adjacent genes. This detailed genetic study showed that the variation in growth habit determined by the VRN1 gene was completely linked to only two genes. Although the possibility that additional genes would be found in the two current gaps and unsequenced regions of our T. monococcum physical maps cannot be ruled out, this seems unlikely based on the comparative studies with rice and sorghum and the absence of any additional genes in the 324-kb of wheat sequence between CYB5 and CYS.

The genetic data reduced the problem of the identification of VRN1 to the question of which of the two MADS-box genes was the correct candidate. However, since no recombination was found between AGLG1 and AP1 it was not possible to answer this question based on the available genetic results. Therefore, the relationship between AGLG1 and AP1 with MADS-box genes from other species was established as a first step to predict their function from the known function of the related genes.

Phylogenetic Relationships of the VRN1 Candidate Genes

The similarity between the wheat AP1 gene and the Arabidopsis meristem identity genes AP1, CAL, and FUL provided a first indication that the wheat AP1 gene was a good candidate for VRN1. These Arabidopsis genes are expressed in the apices and are required for the transition between the vegetative and reproductive phases (34). The triple Arabidopsis mutant ap1-cal-ful never flowers under standard growing conditions (34). In wheat, the VRN1 gene is also responsible, directly or indirectly, for the transition between vegetative and reproductive apices. This transition is greatly accelerated by vernalization in the wheat plants carrying the vrn1 allele for winter growth habit. Therefore, it is reasonable to speculate that the sequence similarity between the wheat AP1 gene and the Arabidopsis meristem identity genes may indicate similar functions. An evolutionary change in the promoter region of AP1 may be sufficient to explain the regulation of AP1 by vernalization in wheat (see model below).

The close relationship of wheat AGLG1 to members of the AGL2 subgroup suggested that AGLG1 was a less likely candidate for VRN1 than AP1 because transcripts from genes included in this group are usually not observed in the apices in the vegetative phase (25). Expression of Arabidopsis AGL2, AGL4 and AGL9 begins after the onset of expression of floral meristem identity genes but before the activation of floral organ identity genes suggesting that members of the AGL2 lade may act as intermediaries between the meristem identity genes and the organ identity genes (35-37). This seems to be valid also for OSMADSi, which is more closely related to AGLG1 than the Arabidopsis members of the AGL2 lade. In situ hybridization experiments of young rice inflorescences with OsMADS1, showed strong hybridization signals in flower primordia but not in other tissues (38).

If the functions of wheat AP1 and AGLG1 were similar to the function of the related genes from Arabidopsis, the initiation of transcription of AP1 should precede the initiation of transcription of AGLG1 in wheat.

Transcription Profiles of the VRN1 Candidate Genes

RT-PCR experiments using RNA samples from vernalized apices showed transcription of AP1 but not of AGLG1 (FIG. 4A) indicating that transcription of AGLG1 occurs after the initiation of transcription of AP1. The similar timing and order of transcription suggests that the wheat genes might perform similar functions to the related Arabidopsis genes.

It could be argued that any gene in the flowering regulatory pathway would be upregulated by the initiation of flowering caused by the vernalization process. However, the upregulation of AP1 transcription in the leaves by vernalization (FIG. 4B) indicated a more direct role of the vernalization pathway in the regulation of wheat AP1 gene. Four additional characteristics of the transcription profile of AP1 paralleled the predicted expression of a vernalization gene. First, vernalization was required to initiate AP1 transcription in the plants with winter growth habit but not in the plants with spring growth habit. Second, AP1 transcription was initiated only after two weeks in the cold room, and a minimum of two weeks of vernalization is required by many winter wheat varieties to produce any significant acceleration of flowering (39). Third, the progressive increase of AP1 transcripts after the second week of vernalization (FIG. 4B) is consistent with the progressive effect of the length of the vernalization period in the acceleration of flowering time (39). Finally, a high level of AP1 transcripts was observed after the plants were moved from the cold to room temperature indicating that AP1 is not just a cold stress induced gene.

Allelic Variation

No differences were found in the AGLG1 coding region or in its 5′ and 3′ regions between T. monococcum accessions G2528 (Vrn1) and DV92 (vrn1) confirming that AGLG1 was not a good candidate to explain the observed differences in growth habit.

Although no differences were detected in the AP1 coding sequences and 3′ region, the spring and winter accessions differed in their promoter sequence. The first 600-bp upstream from the start codon were identical among the four genotypes analyzed in this study except for a 20-bp deletion located close to the start of transcription and adjacent to a putative MADS-box protein binding site (CArG-box) in G2528 (40) (FIG. 4). Two additional overlapping deletions were discovered in the same region of the promoter in spring accessions of cultivated T. monococcum (FIG. 11) and a 1-bp deletion was found by sequencing accession PI503874 (SEQ ID NO:17). The presence of a putative CArG-box in this region suggests the possibility that a trans-acting factor may bind to this site and repress AP1 transcription until vernalization occurs. This is similar to the case for FLC in Arabidopsis, which was recently shown to bind to MADS-box gene SOC1 and repress its transcription prior to vernalization (41).

A Model for the Regulation of Flowering by Vernalization in Wheat

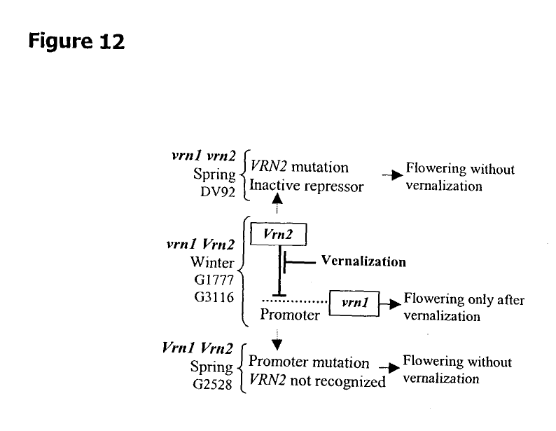

The results presented in this study can be included in an integrated model (FIG. 12) based on the known epistatic interactions between VRN1 and VRN2 (2) and the available information about the evolution of the vernalization requirement in the Triticeae. The significant epistatic interactions observed between VRN1 and VRN2 indicate that these two genes act in the same pathway (2). According to the model presented here (FIG. 12), VRN2 codes for a dominant repressor of flowering that acts directly or indirectly to repress VRN1. As the vernalization process reduces the abundance of the VRN2 gene product, VRN1 transcription gradually increases leading to the competence to flower (FIG. 13, center).

The growth habit of plants homozygous for the recessive vrn2 allele for spring growth habit (FIG. 12, upper panel) is independent of variation at the VRN1 locus (2). According to this model, the vrn2 allele represents a null or defective repressor that cannot interact with the VRN1 promoter. Therefore, variation in the promoter of the VRN1 gene would have no effect on flowering time in homozygous vrn2 plants. This can be illustrated by the expression pattern of AP1 in T. monococcum DV92 (vrn1 vrn4). In this genotype, the initiation of AP1 transcription in leaves and apices did not require vernalization in spite of the presence of a recessive vrn1 allele. This result indicated that the VRN1 gene acts downstream of VRN2(FIG. 12).

Conversely, plants homozygous for the Vrn1 allele for spring growth habit showed no significant effects of the VRN2 gene on flowering time (2). According to the model in FIG. 12 (lower panel), the VRN2 repressor will have no effect on flowering in genotypes carrying the Vrn1 allele because of the lack of the recognition site in the VRN1 promoter region. This part of the model can be used to explain the AP1 expression profile of G2528 (Vrn1 Vrn2). In this genotype, transcription of AP1 in leaves and apices is initiated without a requirement for vernalization in spite of the presence of an active VRN2 repressor. This suggested that the active repressor could not interact with the G2528 AP1 promoter region, possibly because of the presence of the 20-bp deletion.

This model also provides an explanation for the parallel evolution of VRN1 spring alleles in three different Triticeae lineages. A vernalization gene with a dominant spring growth habit has been mapped in the same map location in diploid wheat (1), barley (42), and rye (43). Most of the wild Triticeae have a winter growth habit suggesting that the recessive vrn1 allele is the ancestral character (44-46). This is also supported by the fact that it is unlikely that a vernalization requirement would be developed independently at the same locus in the three different lineages from an ancestral spring genotype. According to the model presented here, independent mutations in the promoter regions of winter wheat, barley, and rye genotypes have resulted in the loss of the recognition site of the VRN2 repressor (or an intermediate gene) and therefore, in a dominant spring growth habit (Vrn1 allele). Since this is a loss rather of a gain of a new function it is easier to explain its recurrent occurrence in the different Triticeae lineages.

In summary, this invention presents the delimitation of the candidate genes for Vrn1 to AP1 and AGLG1 by a high-density genetic map, and the identification of AP1 as the most likely candidate based on its similar sequence to meristem identity genes, its transcription profile, and its natural allelic variation. The model is presented to integrate the results from this study with the previous knowledge about the epistatic interactions between vernalization genes and the evolution of vernalization in the Triticeae.

The following references cited herein are hereby incorporated by reference in their entirety.

1. Dubcovsky, J., Lijavetzky, D., Appendino, L. & Tranquilli, G. (1998) Theor. Appl. Genet. 97, 968-975.

2. Tranquilli, G. E. & Dubcovsky, J. (1999) J. Hered. 91, 304-306.

3. Law, C. N., Worland, A. J. & Glorgi, B. (1975) Heredity 36, 49-58.

4. Galiba, G., Quarrie, S. A., Sutka, J. & Morgounov, A. (1995) Theor. Appl. Genet. 90, 1174-1179.

5. Iwaki, K., Nishida, J., Yanagisawa, T., Yoshida, H. & Kato, K. (2002) Theor. Appl. Genet. 104, 571-576.

6. Barrett, B., Bayram, M. & Kidwell, K. (2002) Pl. Breed. 121, 400-406.

7. Kato, K., Miura, H. & Sawada, S. (1999) Genome 42, 204-209.

8. Kato, K., Kidou, S., Miura, H. & Sawada, S. (2002) Theor. Appl. Genet. 104, 1071-1077.

9. Michaels, S. D. & Amasino, R. M. (1999) Plant Cell 11, 949-956.

10. Sheldon, C. C., Rouse, D. T., Finnegan, E. J., Peacock, W. J. & Dennis, E. S. (2000) Proc Nat Acad Sci USA 97, 3753-3758.

11. Johanson, U., West, J., Lister, C., Michaels, S., Amasino, R. & Dean, C. (2000) Science 290, 344-347.

12. Goff, S. A., Ricke, D., Lan, T. H., Presting, G., Wang, R. L., Dunn, M., Glazebrook, J., Sessions, A., Oeller, P., Varma, H., Hadley, D., Hutchinson, D., Martin, C., Katagiri, F., Lange, B. M., Moughamer, T., Xia, Y., Budworth, P., Zhong, J. P., Miguel, T., Paszkowski, U., Zhang, S. P., Colbert, M., Sun, W. L., Chen, L. L., Cooper, B., Park, S., Wood, T. C., Mao, L., Quail, P., Wing, R., Dean, R., Yu, Y. S., Zharkikh, A., Shen, R., Sahasrabudhe, S., Thomas, A., Cannings, R., Gutin, A., Pruss, D., Reid, J., Tavtigian, S., Mitchell, J., Eldredge, G., Scholl, T., Miller, R. M., Bhatnagar, S., Adey, N., Rubano, T., Tusneem, N., Robinson, R., Feldhaus, J., Macalma, T., Oliphant, A. & Briggs, S. (2002) Science 296, 92-100.

13. Wicker, T., Stein, N., Albar, L., Feuillet, C., Schlagenhauf, E. & Keller, B. (2001) Plant J. 26, 307-316.

14. SanMiguel, P., Ramakrishna, W. Bennetzen, J. L., Busso, C. S. & Dubcovsky, J. (2002) Functional and Integrative Genomics 2, 70-80.

15. Dubcovsky, J., Ramakrishna, W., SanMiguel, P., Busso, C., Yan, L., Shiloff,. B. & Bennetzen, J. (2001) Plant Physiol. 125, 1342-1353.

16. Ramakrishna, W., Dubcovsky, J., Park, Y. J., Busso, C. S., Emberton, J., SanMiguel, P. & Bennetzen, J. L. (2002) Genetics 162, 1389-1400.

17. Dubcovsky, J., Galvez, A. F. & Dvorak, J. (1994) Theor. Appl. Genet. 87, 957-964.

18. Lijavetzky, D., Muzzi, G., Wicker, T., Keller, B., Wing, R. & Dubcovsky, J. (1999) Genome 42, 1176-1182.

19. Zhang, H.-B., Choi, S., Woo, S. S., Li, Z. & Wing, R. A. (1996) Mol Breeding 2, 11-24.

20. Woo, S. S., Jiang, J., Gill, B. S., Paterson, A. H. & Wing, R. A. (1994) Nucleic Acid Research 22, 4922-4931.

21. Kumar, S., Tamura, K. & Nei, M. (1994) Comput Appl Biosci 10, 189-191.

22. Yan, L., Echenique, V., Busso, C., SanMiguel, P., Ramakrishna, W., Bennetzen, J. L., Harrington, S. & Dubcovsky, J. (2002) Mol. Gener. Genomics 268, 488-499.

23. Livak, K. J. & Schmittgen, T. D. (2001) Methods 25, 402-408.

24. Ng, M. & Yanofsky, M. F. (2001) Nat Rev Genet 2, 186-195.

25. Johansen, B., Pedersen, L. B., Skipper, M. & Frederiksen, S. (2002) Mol. Phylogenet. Evol. 23, 458-480.

26. Murai, K., Murai, R. & Ogihara, Y. (1997) Genes Genet Syst 72, 317-321.

27. Murai, K., Takumi, S., Koga, H. & Ogihara, Y. (2002) Plant J. 29, 169-181.

28. Schmitz, J., Franzen, R., Ngyuen, T. H., Garcia-Maroto, F., Pozzi, C., Salamini, F. & Rohde, W. (2000) Pl. Mol. Biol. 42, 899-913.

29. Gardner, J. S., Hess, W. M. & Trione, E. J. (1985) Amer. J. Bot. 72, 548-559.

30. Bennett, M. D. & Smith, J. B. (1991) Phil Trans Roy Soc London B334, 309-345.

31. Stein, N., Feuillet, C., Wicker, T., Schlagenhauf, E. & Keller, B. (2000) Proc Nat Acad Sci USA 97, 13436-13441.

32. Dvorak, J. & Chen, K.-C. (1984) Genetics 106, 325-333.

33. Lukaszewski, A. J. & Curtis, C. A. (1993) Theor. Appl. Genet. 84, 121-127.

34. Ferrandiz, C., Gu, Q., Martienssen, R. & Yanofsky, M. F. (2000) Development 127, 725-734.

35. Flanagan, C. A. & Ma, H. (1994) Pl. Mol. Biol. 26, 581-595.

36. Savidge, B., Rounsley, S. D. & Yanofsky, M. F. (1995) Plant Cell 7, 721-733.

37. Mandel, M. A. &Yanofsky, M. F. (1998) Sex Plant Reprod 11, 22-28.

38. Chung, Y. Y., Kim, S. R., Finkel, D., Yanofsky, M. F. & An, G. H. (1994) Pl. Mol. Biol. 26, 657-665.

39. Limin, A. E. & Fowler, D. B. (2002) Annals of Botany 89, 579-585.

40. Tilly, J. J., Allen, D. W. & Jack, T. (1998) Development 125, 1647-1657.

41. Hepworth, S. R., Valverde, F., Ravenscroft, D., Mouradov, A. & Coupland, G. (2002) EMBO J. 21, 4327-4337.

42. Laurie, D. A., Pratchett, N., Bezant, J. H. & Snape, J. W. (1995) Genome 38, 575-585.

43. Plaschke, J., Börner, A., Xie, D. X., Koebner, R. M. D., Schlegel, R. & Gale, M. D. (1993) Theor. Appl. Genet. 85, 1049-1054.

44. Kihara, H. & Tanaka, M. (1958) Preslia 30, 241-251.

45. Halloran, G. M. (1967) Genetics 57, 401-407.

46. Goncharov, N. P. (1998) Euphytica 100, 371-376.

We claim:

1. A recombinant AP1 promoter sequence wherein said AP1 promoter sequence hybridizes to the nucleic acid molecule of SEQ ID NO:12 or the complement thereof under high stringency conditions, wherein said high stringency conditions consist of hybridization to filter-bound DNA in 5×SSC, 2% sodium dodecyl sulfate (SDS), 100 ug/ml single stranded DNA at 55-65° C., and washing in 0.1×SSC and 0.1% SDS at 60-65°.

2. The recombinant AP1 promoter of claim 1 wherein said promoter sequence lacks all or a portion of the CarG box (SEQ ID NO: 23) located at positions −162 to −172 upstream from the start codon of SEQ ID NO: 12 or comprises one or more deletions in SEQ ID NO:12 as depicted in SEQ ID NO:14, SEQ ID NO:15, SEQ ID NO:16 and SEQ ID NO:17.

3. The recombinant AP1 promoter of claim 1 wherein said AP1 promoter is operably linked to a heterologous protein encoding sequence.

4. The recombinant AP1 promoter of claim 2 wherein said AP1 promoter is operably linked to a heterologous protein encoding sequence.

5. The recombinant AP1 promoter of claim 3 wherein said heterologous protein is an AP1 protein encoding sequence.

6. The recombinant AP1 promoter of claim 4 wherein said heterologous protein is an AP1 protein encoding sequence.

7. The recombinant AP1 promoter of claim 5 wherein said AP1 protein coding sequence hybridizes under high stringency conditions to a nucleic acid selected from the group consisting of SEQ ID NO:6, SEQ ID NO:7, SEQ ID NO:18, SEQ ID NO:22 and the nucleic acid encoding the AP1 protein depicted in SEQ ID NO:19, SEQ ID NO:20, SEQ ID NO:21, SEQ ID NO:8 or SEQ ID NO:9.

8. The recombinant AP1 promoter of claim 6 wherein said AP1 protein coding sequence hybridizes under high stringency conditions to a nucleic acid selected from the group consisting of SEQ ID NO:6, SEQ ID NO:7, SEQ ID NO:18, SEQ ID NO:22 and the nucleic acid encoding the AP1 protein depicted in SEQ ID NO:19, SEQ ID NO:20, SEQ ID NO:21, SEQ ID NO:8 or SEQ ID NO:9.

9. A vector comprising the recombinant AP1 promoter of claim 1.

10. A vector comprising the recombinant AP1 promoter of claim 2.

11. A vector comprising the recombinant AP1 promoter of claim 3.

12. A cell comprising the vector of claim 9.

13. A cell comprising the vector of claim 10.

14. A cell comprising the vector of claim 11.

15. The cell of claim 12 wherein said cell is a plant cell.

16. The cell of claim 13 wherein said cell is a plant cell.

17. The cell of claim 14 wherein said cell is a plant cell.

18. A transgenic plant comprising the recombinant AP1 promoter of claim 1.

19. The transgenic plant of claim 18 wherein said plant is selected from the group consisting of wheat, barley, rye, oats, and forage grasses.

20. Seed from the transgenic plant of claim 19.

21. A transgenic plant comprising the nucleic acid of claim 2.

22. Seed from the transgenic plant of claim 22.

23. The transgenic plant of claim 21 wherein said plant is selected from the group consisting of wheat, barley, rye, oats, and forage grasses.

24. A transgenic plant comprising the nucleic acid of claim 3.

25. Seed from the transgenic plant of claim 24.

26. The transgenic plant of claim 25 wherein said plant is selected from the group consisting of wheat, barley, rye, oats, and forage grasses.

28. A method for altering a plant's response to vernalization, the method comprising: transforming a plant or plant tissue with a genetic construct comprising a recombinant AP1 promoter as in claim 3 and expressing the genetic construct in said plant to alter said plant's response to vernalization.

29. A method for altering a plant's response to vernalization, the method comprising: transforming a plant or plant tissue with a genetic construct comprising a recombinant AP1 promoter as in claim 4 and expressing the genetic construct in said plant to alter said plant's response to vernalization.

30. The method of claim 28 wherein said heterologous protein sequence is an AP1 sequence.

31. The method of claim 29 wherein said heterologous protein sequence is an AP1 sequence.

32. The method of claim 28, wherein the plant is selected from the group consisting wheat, barley, rye, oats, and forage grasses.

33. The method of claim 29, wherein the plant is selected from the group consisting wheat, barley, rye, oats, and forage grasses.

34. The method of claim 28 wherein said AP1 promoter sequence is selected from the group consisting of SEQ ID NO:13, SEQ ID NO:14, SEQ ID NO:15, SEQ ID NO:16 and SEQ ID NO:17.

35. The method of claim 29 wherein said AP1 promoter sequence is selected from the group consisting of SEQ ID NO:13, SEQ ID NO:14, SEQ ID NO:15, SEQ ID NO:16 and SEQ ID NO:17.

36. A recombinant AP1 promoter comprising a nucleotide sequence selected from the group consisting of SEQ ID NO:9, SEQ ID NO:10, SEQ ID NO:11, SEQ ID NO:12 and SEQ ID NO:13, SEQ ID NO:14, SEQ ID NO:15, SEQ ID NO:16, SEQ ID NO:17 and SEQ ID NO:23.

37. A molecular marker for Vrn1 derived from a gene selected from the group of genes depicted in FIG. 1.
